(19) United States
(12) Patent Application Publication
(10) Pub. No.: US 2014/0088567 A1
(43) Pub. Date: Mar. 27, 2014

(54) Compositions and Methods for Removing Ascitic Fluid from the Abdomen

(71) Applicants: Gary F. Nieman, Manlius, NJ (US); Kathleen P. Snyder, North Syracuse, NY (US); Louis Gatto, Cortland, NY (US); Shreyus Roy, Syracuse, NJ (US); Benjamin Sadowitz, Camillus, NJ (US)

(72) Inventors: Gary F. Nieman, Manlius, NJ (US); Kathleen P. Snyder, North Syracuse, NY (US); Louis Gatto, Cortland, NY (US); Shreyus Roy, Syracuse, NJ (US); Benjamin Sadowitz, Camillus, NJ (US)

(73) Assignee: THE RESEARCH FOUNDATION OF STATE UNIVERSITY OF NEW YORK, Albany, NY (US)

(21) Appl. No.: 13/983,575

(22) PCT Filed: Feb. 4, 2012
(86) PCT No.: PCT/US2012/023902

§ 371 Date: Dec. 3, 2013

Related U.S. Application Data
(60) Provisional application No. 61/439,794, filed on Feb. 4, 2011.

(51) Int. Cl. A61M 1/00; A61M 39/08

(52) U.S. Cl. CPC A61M 1/008; A61M 39/08
USPC 604/533; 604/543

(57) ABSTRACT

The present invention features devices that can be used to extract ascites and other fluids from the body with a minimally invasive procedure and, if desired, can also be used to flush the cavity (e.g., the abdominal cavity) from which the fluids were removed. The devices also permit characterization of the fluids in that a surgeon or other health care provider can conveniently obtain a sample of the fluids to assess their composition. The amount of fluid removed from a cavity can also be determined. While the present devices and procedures are not limited to those that bring about any particular physiological response, we believe they will help maintain blood flow and prevent the accumulation of toxins and inflammatory cytokines in bodily cavities, such as the abdominal cavity. We may refer to the devices of the invention as MIST, as they allow for minimally invasive suction and treatment.

### CROSS-REFERENCE TO RELATED APPLICATIONS

This application claims the benefit of the filing date of U.S. provisional application No. 61/439,794, which was filed Feb. 4, 2011. The content of this provisional application is hereby incorporated by reference herein in its entirety for any U.S. application that claims the benefit of the filing date of the present application and any U.S. patent(s) that issue therefrom.

### FIELD OF THE INVENTION

The present invention relates to compositions and methods that can be used to remove harmful fluids that accumulate in the abdominal cavity in the event of sepsis and other medical conditions and, more particularly, to devices that allow the identification, collection, dilution and/or removal of ascitic fluid from the abdomen of a subject with minimally invasive surgical techniques.

### BACKGROUND

The acute respiratory distress syndrome (ARDS) affects nearly 88,000 patients per year and, in spite of intensive research and improved mechanical ventilation strategies, still claims the lives of nearly 30% of the patients it afflicts (Goss et al., Crit. Care Med. 31(6):1607-1611 (2003)). ARDS can be caused by either a primary or secondary mechanism; primary ARDS is caused by direct injury to the lung such as aspiration or pneumonia whereas secondary ARDS is caused by systemic inflammation following severe injury such as trauma, hemorrhage, or sepsis. The reason that secondary ARDS occurs in some patients and not others, with seemingly similar injuries, is unknown. Since the gut is preferentially injured in hemorrhage and sepsis, it has been hypothesized that increased gut epithelial permeability resulting in bacterial translocation is the “motor” of secondary ARDS (Swank and Deitch, World J. Surg. 20(4):411-417 (1996)). However, multiple studies have suggested that this is not the case (Plotz et al., Intensive Care Med. 30(10):1865-1872 (2004); Ince, Crit. Care 9 Suppl 4:S13-19 (2005); Imai et al., JAMA 289(16):2104-2112 (2003); and David et al., Crit. Care 10(4):R100 (2006)). Another theory proposes that toxic mesenteric lymph is the “motor” of ARDS. This gut/lymph hypothesis postulates that toxic mediators from damaged intestine are picked up in the lymph and then delivered directly to the lung through the superior vena cava (Adams et al., Am. Surg. 66(10):905-912 (2000); Magnotti et al., Ann. Surg. 228(4):518-527 (1998); and Deitch et al., Front Biosci. 11:520-528 (2006)).

Currently, there is no clinical treatment strategy to directly address microcirculatory or endothelial dysfunction. Aggressive fluid resuscitation, along with vasoactive medications to optimize oxygen delivery remains the standard of care. Recent studies have shown the beneficial effects of flushing the peritoneum of shocked rats with infusions of dialysis fluid in a new therapeutic modality called direct peritoneal resuscitation (DPR). This novel therapy has improved microcirculation, reduced endothelial dysfunction, and decreased organ injury in small animal models of hemorrhagic shock (Zakaria el et al., J. Trauma 58(3):499-506 (2005); Zakaria el et al., Am. J. Surg. 186(5):443-448 (2003); Zakaria el et al., J. Am. Coll. Surg. 206(5):970-980 (2008); and Zakaria el et al., Shock 27(4):436-442 (2007)). To our knowledge, DPR has not yet been attempted in a translational setting using large animals.

### SUMMARY

The present invention is based, in part, on our recognition that third-space fluids, including ascites fluid released from capillaries in the gut, can be pro-inflammatory and play a critical role in the pathogenesis of secondary ARDS. We have recognized that ascites mobilizes the inflammatory response in sepsis and contributes to Abdominal Compartment Syndrome (ACS); both sepsis and ACS greatly increase morbidity and mortality (Bailey, Crit. Care 4:23-29 (2000)). Accordingly, we have developed devices that can be used to extract ascites and other fluids from the body with a minimally invasive procedure and, if desired, can also be used to flush the cavity (e.g., the abdominal cavity) from which the fluids were removed. The devices also permit characterization of the fluids in that a surgeon or other health care provider can conveniently obtain a sample of the fluids to assess their composition. The amount of fluid removed from a cavity can also be determined. While the present devices and procedures are not limited to those that bring about any particular physiological response, we believe they will help maintain blood flow and prevent the accumulation of toxins and inflammatory cytokines in bodily cavities, such as the abdominal cavity. We may refer to the devices of the invention as MIST, as they allow for minimally invasive suction and treatment.

The devices include a central tube, which may contain either a single lumen or multiple lumens, and the devices can have either a single port or multiple ports for supplying or removing fluids through the lumen(s). In the simplest configuration, the device has one port and one lumen. The port is configured to connect to a vacuum source for fluid removal and may also be configured to receive a supply of fluid. The lumen carries either the fluid being removed or the fluid being supplied in order to lavage or treat the targeted cavity. In another configuration, the device has one port and two lumens. As just described, the single port is configured so it can be connected to a vacuum source and can also, if desired, be suitable for receiving a fluid supply. Distal to the port, the central tube contains two lumens. Each lumen may extend directly from the port, or the port can be initially connected to a single lumen that then bifurcates. Devices with one port and two lumens can include a switch that directs fluid flow into one lumen or the other. For example, to remove fluid from the abdominal cavity, a user may direct the fluid toward a fluid removal lumen that is connected through a manifold to a plurality of catheters. To supply fluid to the abdominal cavity, a user may direct the fluid to an infusion lumen. Where the device includes two ports, they may be either essentially identical or customized for either fluid supply or removal. For example, the device can include a first port for fluid removal (e.g., ascites removal) and a second port for fluid injection or supply (e.g., a resuscitation fluid used to lavage the targeted body cavity). Each port can be connected to a lumen. For example, the fluid removal port can be connected to a fluid removal lumen that, in turn, connects through a manifold to a plurality of catheters whose distal ends can be distributed throughout the targeted cavity. The fluid supply port can be connected to a fluid supply lumen, which may exit the central tube without further bifurcation to supply fluid to the targeted cavity. Where there are multiple ports, the ports can be located adjacent one another (i.e., in the same vicinity of the device) or on separate arms of the device. For example, a first fluid removal port and a second fluid supply port can both be located at the proximal end of the central tube. Multiple ports can also be located together on an arm that extends from the central tube (e.g., at the proximal end of an auxiliary tube that branches from the central tube). Alternatively, a first port for fluid removal can be positioned at the proximal end of the auxiliary tube and a second port for fluid supply can be positioned at the proximal end of the central tube and vice versa. Distancing one port from one another may improve ease of use.

The devices can be used to remove unwanted fluid produced by the patient's body as well as to remove fluids introduced from an external source. For example, the devices can be configured to remove ascites with suction and deliver a peritoneal resuscitation fluid to an injured intestine. The fluid removal and delivery can be carried out simultaneously or sequentially to essentially flush the bodily cavity. For example, a device as described herein can be positioned and ascites or other fluids can be withdrawn before delivering and subsequently aspirating a resuscitation fluid. As noted, the device can include a central tube with a single lumen or multiple lumens. Thus, various fluids can be delivered through the same or different lumens. In some embodiments, the device is configured with a single lumen running through the central tube and that same lumen can be used to deliver a resuscitation fluid as well as to remove the patient's own fluids. In other embodiments, the device is configured with multiple (e.g., dual) lumens running within the central tube, and ascites and other fluids would be aspirated through a first fluid removal lumen and a resuscitation fluid would be delivered through a second fluid supply lumen. A lumen is considered to be “within” the central tube when some or all of the lumen runs within the outer wall of some or all of the central tube. Alternatively, resuscitation fluids can be delivered through a separate device (e.g., tubing that is not physically connected to the device described herein for aspirating bodily fluids). Further, the delivery of resuscitation fluid and subsequent aspiration can be carried out more than once (e.g., 2-5 times), effecting multiple rounds of fluid flushing.

Accordingly, in one aspect, the invention features a medical device for removing fluid from a bodily cavity. The device includes a central tube having a proximal end configured as a fluid removal port that receives an external vacuum source and a distal end comprising a manifold (or comprising a connector to a non-integral manifold) from which a plurality of adaptors extend. The adaptors have tips to which one or more catheters suitable for placement in the bodily cavity can be attached.

Unless the context clearly indicates otherwise, we use the term “proximal” to refer to the portion of the device that is nearer the user (e.g., the surgeon) and further from the subject (e.g., the patient being treated). The term “distal” refers to the portion of the device that is nearer the subject and further from the user. As in the art generally, the terms proximal and distal may be used herein to indicate the relative positions of components of the device.

The central tube can be rigid or semi-rigid along some or all of its length by virtue of being constructed from or including a rigid or semi-rigid material (e.g., aluminum, steel, a metal alloy, or a plastic or polymer such as polyurethane), and the manifold can have a diameter that is at least or about as large as the combined diameters of the plurality of adaptors. In some instances, for example, where the manifold includes openings around a peripheral wall and on a bottom wall, the diameter of the manifold may be less than the combined diameters of the plurality of adaptors extending therefrom. Whether rigid or semi-rigid, the central tube can have one or more of the following characteristics: an outer diameter of about 5-20 millimeters; a wall thickness of about 1-3 millimeters; an inner diameter of about 2-19 millimeters; and a length of about 5-20 centimeters. For the avoidance of doubt, the diameter of the central tube is measured on a plane perpendicular to the long axis of the central tube; a cross-section taken perpendicular to the long axis.

In cross-section, the central tube can be cylindrical or polygonal. As described further herein, the manifold can be an integral part of the central tube or a distinct component of the device that is attached or affixed to the central tube. In either case, the manifold, in cross-section, can also be cylindrical or polygonal. In either case, the manifold can have, around the circumference of a peripheral surface, three to eight openings (i.e., 3, 4, 5, 6, 7, or 8 openings) from which three to eight adaptors (i.e., 3, 4, 5, 6, 7, or 8 adaptors) respectively extend. When the manifold is distinct from the central tube, the manifold can include one or more of a top surface, a peripheral surface, and a bottom surface, and can have one or more of the following characteristics: an opening on the top surface to which the central tube can be attached or affixed; a diameter of about 5-50 (e.g. about 15-50) mm; a peripheral wall height of about 10-30 mm; a plurality of openings in the peripheral wall from which adaptors extend; and/or a plurality of openings on the bottom surface from which adaptors extend. When the manifold is an integral part of the central tube (e.g., where the manifold is composed of openings in the distal portion of the central tube), the central tube can have a bottom surface and a plurality of openings in the peripheral wall or bottom surface from which adaptors extend. The openings in the surface of the central tube constitute the manifold We use the term “manifold” to mean a part that distributes fluid to multiple channels. Thus, the portion of the device in which fluid is transitioned from a single lumen to multiple lumens is the manifold. The adaptors extend from the manifold; the adaptors are distal to the manifold.

To facilitate the attachment of catheters to the adaptors, the adaptors can be spaced apart and/or constructed to various lengths. For example, where the adaptors extend from the bottom surface of the manifold or the distal end of the central tube (e.g., the distal peripheral wall or distal bottom wall), they can vary in length relative to one another and/or be angled to project away from the long axis of the central tube. For example, the adaptor can be formed as a rigid tube that initially extends along the long axis of the central tube and then turns to extend at an angle (e.g., a right angle) away from the line of the long axis. When viewed from the end, adaptors in this configuration would appear to radiate outward from the central long axis of the device. Similarly, when adaptors extend from around the peripheral wall of the central tube, they would appear to radiate outward from the central long axis of the device.

In another aspect, the invention features a catheter configured to be attached at its proximal end to an adaptor of the device described herein. The catheter includes an open proximal end configured to receive the adaptor tip, a peripheral wall running the length of the catheter, and an open distal end. At or near the distal end, the catheter includes at least one inflated or inflatable balloon that encircles an outer portion of the peripheral wall of the catheter and thereby helps suspend the open distal end away from tissue. Suspending the distal end away from tissue is expected to maximize fluid removal by preventing clogging of the distal tip of the catheter. In some embodiments, the device includes a plurality of such catheters attached to the respective tips of the plurality of adaptors. In some embodiments, the devices of the invention can include an external sleeve concentric to the central tube into which the plurality of catheters can be retracted. At least one of the plurality of catheters can be perforated along its length, and at least one of the plurality of catheters can have an inflatable balloon around the periphery of its distal end. The catheter tubing can include any physiologically acceptable material. For example, the catheters can include polyurethane and have one or more of the following characteristics: an outer diameter of about 5-10 mm; a thickness of about 1-2 mm; an inner diameter of about 3-9 mm; a vacuum rating of about 600-800 mmHg; and a length of about 5-25 cm.

The central tube can include one lumen having a proximal end configured to receive an external fluid source and a distal end having at least one opening for introducing the external fluid into the bodily cavity. Moreover, such a lumen can be parallel to, adjacent to, or concentric inside or outside of another lumen. For example, the fluid supply lumen can be parallel to and concentric inside the fluid removal lumen. The fluid supply lumen can be positioned relative to the central tube such that the distal end of the fluid supply lumen extends beyond the manifold (e.g., beyond the distal end of the central tube and any adaptors extending therefrom. To facilitate the introduction of an external fluid to the device and subsequently to the patient, the fluid supply lumen can include a valve proximal to its proximal end. For example, the valve can include threads for mating with a threaded syringe containing the external fluid or can include a means for docking with tubing from a bag (e.g. a bag of the type used to deliver intravenous fluids). This area of the device (i.e., the proximal end of the fluid supply lumen and the fluid supply port) can also be fitted with a stopcock for regulating the flow of external fluid into the device.

As noted, the present devices can include multiple lumens. In one embodiment, the device has a central tube including a first lumen and a second lumen, the second or fluid supply lumen being aligned with the central tube along the entire length of the second or fluid supply lumen and the first or fluid removal lumen being aligned with the central tube along a distal portion of the first or fluid removal lumen and misaligned with the central tube along a proximal portion of the first or fluid removal lumen. The lumen through which fluids are extracted (e.g., the “first” lumen) can include a sampling port in the vicinity of its proximal end. The surgeon or an assistant can remove a sample of the fluid through the sampling port for analysis.

In another aspect, the invention features methods of treating or reducing the risk of multiple organ dysfunction syndrome (MODS) by (a) providing a patient at risk for MODS; and (b) performing a minimally invasive surgical procedure that removes ascites fluid from the abdominal cavity. These steps can also be carried out to treat or reduce the risk of ARDS, ACS, and sepsis. Any of the methods can further include a step of lavaging the abdominal cavity, and the surgical procedure can be carried out with the devices described herein. More specifically, the methods of the invention can be carried out in a process including the following steps: (a) inserting the manifold, and if already attached to the manifold via adaptors, the plurality of catheters, of a device as described herein through an abdominal incision (and preferably an incision of limited length; compatible with the use of a trocar and laprascopic procedures); (b) if catheters are not already attached to the plurality of adaptors extending from the manifold, attaching the proximal ends of a plurality of catheters to the tips of a plurality of adaptors, optionally using laparoscopic tools; (c) placing a distal end of at least one of the plurality of catheters into an anatomic recess of the abdominal cavity; and (d) applying a negative pressure to the central tube and, more particularly, to the fluid removal port such that the negative pressure is transmitted through the fluid removal lumen, drawing fluid from the body cavity into and through the device.

The step of applying negative pressure to the central tube can be carried out by attaching the fluid removal port at the proximal end of the central tube to a vacuum source (e.g., a vacuum pump or any other negative pressure source). The methods can also include a step, carried out after applying a negative pressure to the central tube, of collecting abdominal fluid removed from the patient via the catheters and fluid removal lumen and, optionally, characterizing the amount and/or content of the fluid. The methods can also include a step, either before or after applying a negative pressure to the central tube, of lavaging the abdominal cavity. The anatomic recess into which a catheter is placed can be the lesser sac, Morrison's pouch, pouch of Douglas, or a pericolic gutter. Lavaging the abdominal cavity can include delivering a sterile, physiologically acceptable fluid solution to the abdominal cavity (e.g., a fluid that is, or that includes, normal saline; the fluid may be buffered (e.g., it may be a buffered saline solution)). The methods can also include the step of administering a therapeutic agent to the bodily cavity (e.g., the abdomen) from which the fluids have been removed. For example, one could administer an antimicrobial agent (e.g., an antibiotic, antiviral, or antifungal agent), a vasoactive agent, an anti-inflammatory agent, or any combination thereof. The methods can also include a step in which the abdominal cavity is inflated with a physiologically acceptable gas to facilitate insertion of the plurality of catheters to the abdominal recesses.

In another aspect, the invention features kits that include the medical devices described herein and instructions for use. For example, a kit can include a medical device, instructions for use, and one or more of: a plurality of catheters adapted for attaching to the tips of the plurality of adaptors of the medical device; a sterile fluid; a syringe configured for attachment to a sampling port of the medical device; a syringe, bag, or other container configured for attachment to a fluid supply port; and a therapeutic agent. The sterile fluid can be suitable for lavaging the abdominal cavity or for delivering the therapeutic agent. At least one of the plurality of catheters can be perforated along its length, and at least one of the plurality of catheters can have an inflatable balloon around the periphery of its distal end. The compositions and methods of the present invention are advantageous in that they can be employed with minimally invasive surgical techniques. They can also provide for both direct peritoneal resuscitation (DPR) and removal of ascites through suction. Modification of the distal ends of the catheters to include a balloon-expanded stent is also advantageous as that feature may reduce the risk that the catheters will be clogged by abdominal adhesion. To our knowledge, no minimally invasive, clinically proven device or technique currently exists that can effectively remove peritoneal fluid from the abdominal cavity during shock as a means to treat or reduce the risk of multiple organ dysfunction syndrome (MODS). Since the surgical procedure is minimally invasive, patients and their physicians may be more willing to employ it sooner, allowing for earlier application and better outcomes.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIGS. 1A and 1B are schematics of a representative device of the invention deployed in vivo. Catheters extending from the adaptor tips extending from the manifold (not shown in detail in this schematic) are placed in various abdominal recesses as seen in the coronal (FIG. 1A) and sagittal sections (FIG. 1B).

FIG. 2 is a photograph of a device of the invention. In the upper part of the photograph is a central tube having a proximal end (to the right) configured to receive an external vacuum source and, at the distal end, a manifold from which adaptors of various lengths extend. In the lower part of the photograph, the central tube is inserted through a trocar and one of the adaptors is attached to a catheter (in this illustration, a BLAKE® drain).

FIG. 3 is a photograph of a device of the invention. This device has one port and one lumen. The manifold (in black) is interposed between the central tube and the adaptors, one of which is attached to a catheter (a BLAKE® drain). The port at the proximal end (to the right) is attached to a tube leading to a vacuum source. Thus, the single port is set up for use as a fluid removal port. It could be connected to a fluid source, in which case it would be set up for use as a fluid supply port. The device is positioned adjacent to a trocar, through which the central tube can be passed when used in minimally invasive surgical procedures.

FIG. 4 is a photograph of the junction between the distal end of the central tube and the manifold from which a plurality of adaptors extend. A portion of a BLAKE® drain, which the surgeon would attach to one of the adaptor tips, is shown along the bottom of the photograph.

FIG. 5 is a photograph of a catheter, commercially available as a BLAKE® drain, enlarged to show the detail along its length.

FIG. 6 is an illustration of a device of the invention.

FIG. 7 is an illustration of a portion of a device of the invention including a manifold, the distal tip of a fluid supply lumen, and radially positioned adaptors with tips.

FIG. 8 is an illustration of a device of the invention.

### DETAILED DESCRIPTION

Devices:

The devices of the invention can be used for peritoneal resuscitation and suction removal of ascites. As described above, the devices are configured to remove fluids from bodily cavities and can be used to carry out direct peritoneal resuscitation of the intestine (e.g., with dextrose-based dialysis fluid or any other physiologically acceptable or compatible fluid) with suction removal of ascites. The fluid (e.g., ascites) removal component includes a plurality of adaptors (e.g., 2-10 adaptors) permanently or removably attached to a manifold. The manifold, in turn, can include a port (e.g., a central opening on the top surface) to which a suction generator can be directly attached or to which a central tube containing one or more lumens that interface with a suction generator can be attached. We may use terms such as “suction generator” and “negative pressure device” interchangeably, as both produce a force that pulls fluid through the device, thereby removing it from the patient's body. As noted, the manifold can be an integral part of the central tube, generated in the distal region of the central tube essentially by creating openings in the peripheral wall and/or bottom wall of the central tube. Alternatively, the manifold can be a distinct component removeably attached or permanently affixed to the central tube.

Both the adaptors extending from the manifold and the catheters extending from the adaptor tips can vary in length and diameter, with each parameter varying to accommodate the placement of the catheter's distal ends in various regions of the body (e.g., various abdominal compartments). For example, the length and diameter of a catheter can be varied to accommodate placement in a dependent anatomic recesses of the peritoneal cavity. These recesses include the lesser sac, Morrison's pouch, pouch of Douglas, and the pericolic gutters. Any accumulating fluid can then be removed upon the application of suction. Direct peritoneal resuscitation can be achieved through a reservoir (e.g., a porous, flexible reservoir), which may be permanently or removeably attached to the device and filled with a fluid (e.g., peritoneal dialysis fluid) prior to use. The fluid (e.g., a dialysis fluid) can be delivered simply by gravity. In vitro studies have shown excellent multiphase fluid removal from a closed cavity system using −125 mmHg of negative pressure. As various forms of tubing (e.g., catheters and drains), including commercially available tubing intended for medical and surgical use (e.g., a BLAKE® drain or a drain in the style of a BLAKE® drain) can be attached to a MIST device, we may use the terms tubing, catheter and drain interchangeably to refer to these appendages.

As noted, the term “manifold” refers to the region of the device where a lumen within the central tube joins the plurality of adaptors. The manifold includes openings, which may be radially positioned such that one or more of the adaptors extend laterally from the distal end of the central tube and/or from a bottom surface of the central tube. An advantage of linear extensions is that the device can be more readily passed through a trocar or other surgical guide. However, adaptors extending outward (at an angle from the long axis of the central tube) can include locking joints, allowing them to be deployed outwardly after having passed through the trocar. Where adaptors extend linearly from the distal end of the central tube, they can vary in length, and it is our expectation that such variation will allow the surgeon to more easily attach a catheter (e.g., a BLAKE® drain) to the adaptor.

In some embodiments, the distal ends of the catheters can include an expandable cavity (e.g., a balloon expanded stent) which, when inflated, would help prevent the distal ends of the catheters from coming into direct contact with the patient's abdominal tissue. This, in turn, facilitates fluid removal as the distal ends of the catheters remain free and unblocked by tissue. In other embodiments, the catheters include slits along their length, and this configuration also reduces the risk of impaired fluid flow (e.g., due to clogging or tissue blockage).

The twin goals of peritoneal resuscitation and suction removal can be achieved with single- or dual-lumen devices, but the dual-lumen configuration has certain advantages. For example, a central lumen in the central tube, which we may refer to as a fluid supply lumen, can be used to deliver fluids (e.g., a dextrose-based dialysis fluid) to lavage the abdomen while a surrounding lumen within the central tube but peripheral to the central lumen, which we may refer to as a fluid removal lumen, can be used to aspirate fluids (e.g., ascites) from the abdomen. In other embodiments, the two lumens can be configured differently. For example, they can run side-by-side over at least part of their length in a non-concentric manner. Fluid delivery can be achieved by attaching the distal end of the fluid supply lumen to a catheter in the same or similar way the catheters used for suction removal of fluid are attached to the fluid removal lumen. In one embodiment, the fluid supply port exits the center of the manifold or through the center of the central tube, and adaptors (e.g., for the connection of Blake drains) encircle the fluid supply port where it exits the central tube. The fluid delivery port can be attached to a standard peritoneal dialysis catheter. Catheter-extension from the fluid supply lumen is optional. The fluid passed through the fluid supply lumen may simply transition from the device to the patient's body through an opening or openings in the distal end of the fluid supply lumen.

The central tube can be made from a rigid or semi-rigid material (e.g., a plastic or polymer typically used in surgical devices (e.g., polyurethane)), and it can have a diameter that is at least or about as large as the combined diameters of the plurality of catheters. More specifically, the outer diameter of the central tube can be about 5-20 mm (e.g., 15 mm); the wall, which can be of a uniform or non-uniform thickness, can be about 1-3 mm thick; the inner diameter can be at least or about 2-19 mm (e.g., at least or about 4-5 to 17-19 mm); and the length can be about 5-20 cms. The shape of the manifold can vary, and the central tube and/or manifold may be cylindrical or polygonal. Around the periphery near the distal tip and/or on the distal bottom surface, the manifold will include a plurality of openings from which adaptors extend. For example, 2 to about 7 openings can extend from the peripheral wall and/or 2 to about 7 openings can extend from the bottom surface in line with the central tube. Where the manifold is an integral part of the central tube, the openings for the adaptors can be formed in the peripheral wall of the distal end of the central tube. Where the manifold is an integral part of the central tube, the distal end of the central tube may include a bottom surface perforated by one or more openings. For example, where the device has a single lumen running through the central tube, the distal end of the central tube may include a bottom surface perforated by 2-7 openings from which 2-7 adaptors may extend. Thus, the adaptors may radiate from around the periphery of the distal end of the central tube, thereby extending at an acute angle (e.g., about a 90° angle) from the central tube, or may extend from the bottom of the central tube, thereby extending from the device along roughly the same line as the central tube. Where the manifold is distinct from the central tube, it can have a top surface that interfaces with the distal end of the central tube, a peripheral wall, and a bottom surface. The manifold can also have one or more of the following characteristics: a centrally located opening on the top surface to which the central tube can be attached; a diameter of about 15-50 mm; a peripheral wall height of about 10-30 mm; a plurality of openings in the peripheral wall from which adaptors extend; and/or a plurality of openings on the bottom surface from which adaptors extend. As noted, the manifold and the central tube can have about the same diameter and the adaptors can extend outward from the central line of the device or linearly along the central line of the device.

The adaptors extending from the device can be made of the same material as the central tube and may be more flexible than the central tube. The adaptors can also vary in length from one device to another. Within a given device, the adaptors can also vary in length from one another. For example, where the adaptors extend along the central line of the device, it may be easier to attach a number of catheters when the adaptors are not all the same length.

The catheters attached to the adaptors can be perforated along their length (e.g., they may include slits or openings of other dimensions) and they may include an inflatable balloon. For example, the tubing extending into a subject's bodily cavity may include an inflatable balloon at a point toward the distal tip that inflates around an outer portion of the peripheral wall of the tubing in order to help prevent the tubing from lying immediately next to tissue and becoming clogged. The inflatable balloon can help stabilize the open distal tip of the tubing in the pools of fluid.

Where the materials or components of the device (e.g., the central tube and manifold or the central tube and an adaptor) are joined together, they may connect by a friction fit, and the interfaces may be tapered to facilitate their connection. For example, the central tube may be tapered to fit over or into the manifold, and the distal tips of the adaptors may be tapered to fit over or into the catheters or drains the surgeon will attach to the adaptors. Alternatively, the joints may include an affirmative fastener, such as a snap-lock, or threads for screwing the pieces together.

The catheters can be made from polyurethane or any other flexible material used in surgical devices and tubing, and they may have one or more of the following characteristics: an outer diameter of about 5-10 mm; a thickness of about 1-2 mm; an inner diameter of about 3-9 mm; a vacuum rating of about 600-800 mmHg; and a length of about 5-25 cm.

The central tube can include multiple lumens. For example, the central tube can include a dual lumen, which may be configured such that the two lumens run parallel and adjacent to one another (side-by-side) or one may run inside the other creating a central lumen and a peripheral lumen.

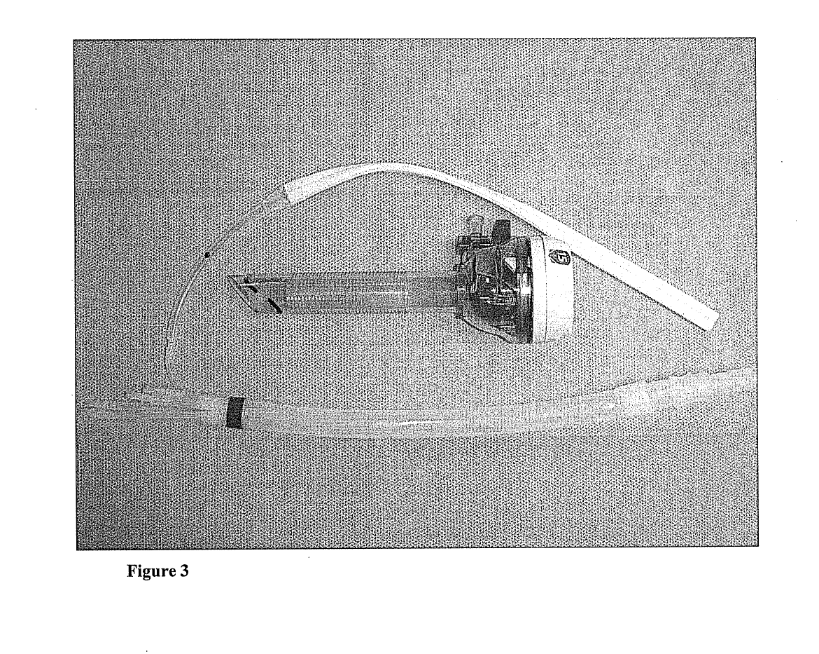

Turning to the photographs of devices we have constructed to date, FIG. 2 shows a MIST device inserted into a Trocar with a BLAKE® drain attached. A second MIST device is shown at the top of the photo. The devices include a central tube having a proximal end (to the right) configured to receive an external vacuum source and a distal end connected to a plurality of adaptors. In this embodiment, the adaptors extend linearly from the manifold and central tube. In FIG. 3, one of the distal adaptors is attached to a BLAKE® drain and the proximal end (to the right) is attached to a tube leading to a vacuum source. The device is positioned adjacent to a trocar, through which the central tube can be passed. In use, the surgeon would make a small incision in the abdomen, and the trocar would be inserted into the peritoneal cavity. MIST would be inserted into the center of the trocar and the trocar would then be removed, leaving only the MIST device in place. If necessary, the device can include a flange that would come to rest near the body wall and the flange could be sutured to the body wall for added stability. Any of these steps can be a step in the treatment methods described below.

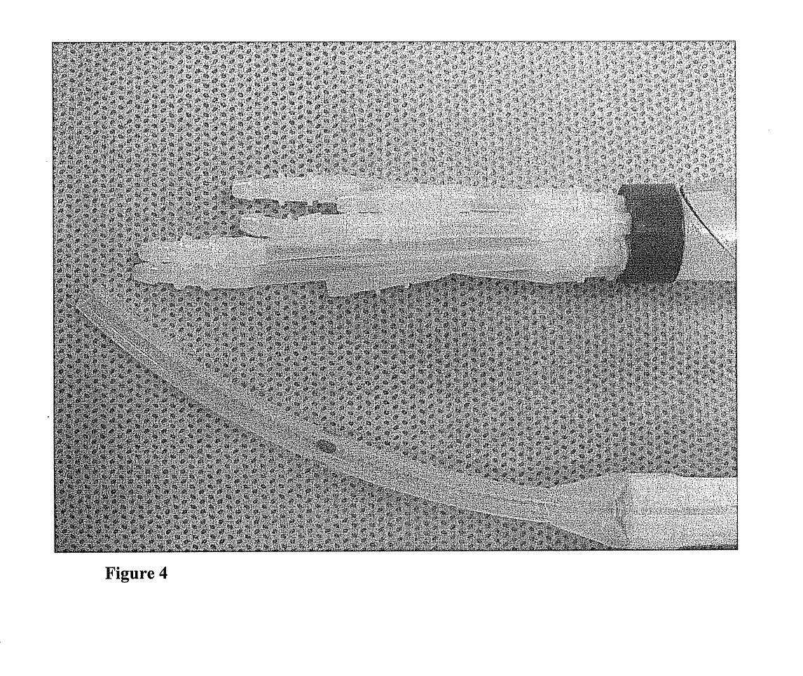

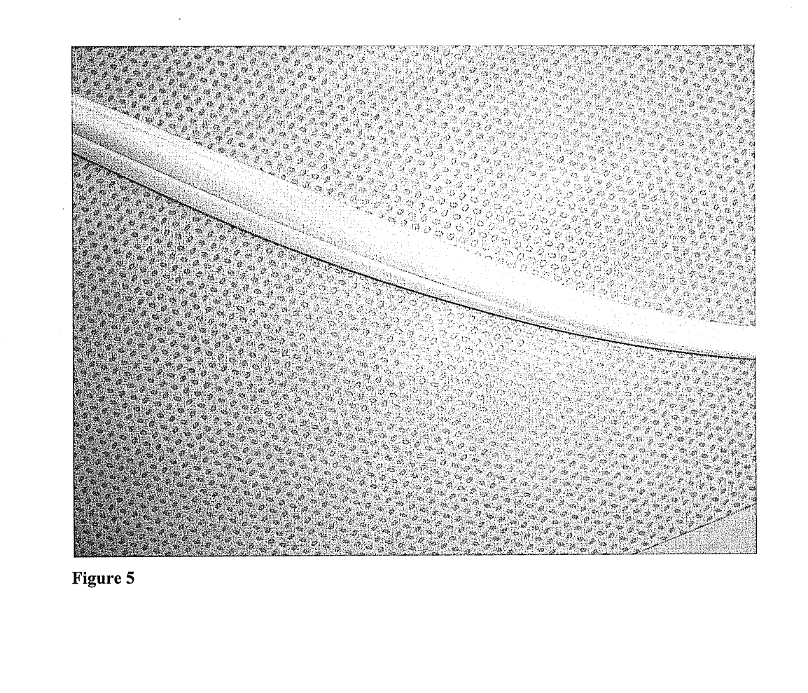

FIG. 4 illustrates the varied lengths of the adaptors extending linearly from the manifold (to the right). The end of a BLAKE drain, which the surgeon would attach to one of the adaptor tips, is shown along the bottom of the photograph. FIG. 5 illustrates the slits that perforate the Blake drain tubing.

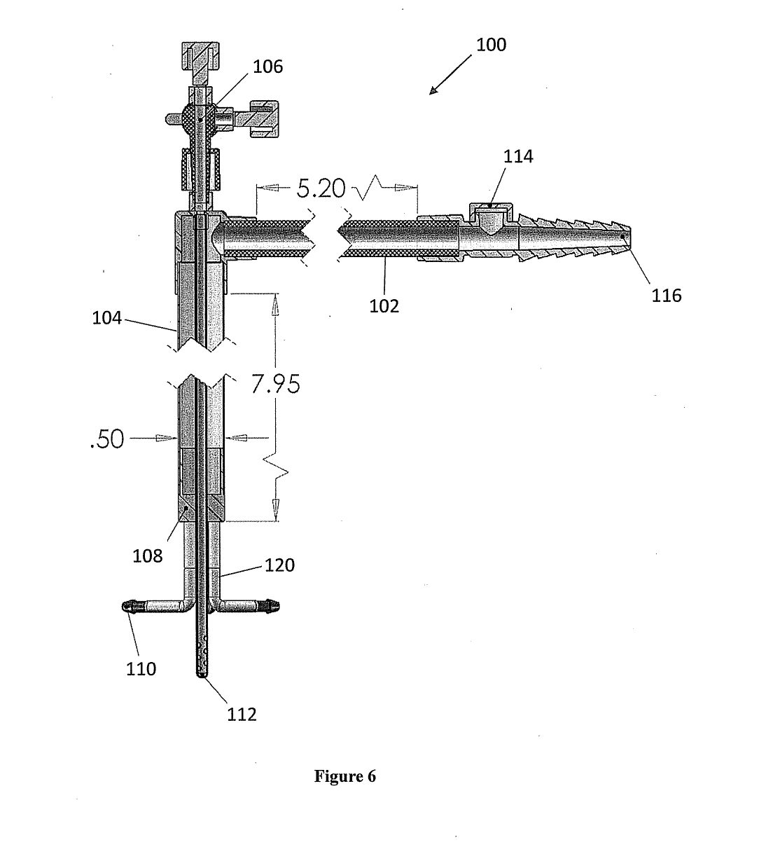

Turning to FIG. 6, device 100 includes a central tube 104 and an auxiliary tube 102. A fluid removal lumen runs through the central tube and auxiliary tube, and a fluid supply lumen runs through the central tube. Fluid removal port 116 connects the fluid removal lumen to a vacuum source, and fluid supply port 106 connects to the fluid supply lumen running through the central tube 104. The distal tip of the fluid supply lumen 112, through which the supplied fluid flows into the patient's body, is seen at the bottom of FIG. 6. The manifold 108 includes openings that connect the fluid removal lumen within the central tube to a plurality of adaptors 120. The adaptor tips 110 are attached to flexible catheters that are positioned within the body cavity as described herein. As fluid is removed through the fluid removal lumen a sample can be obtained at the sampling port 114.

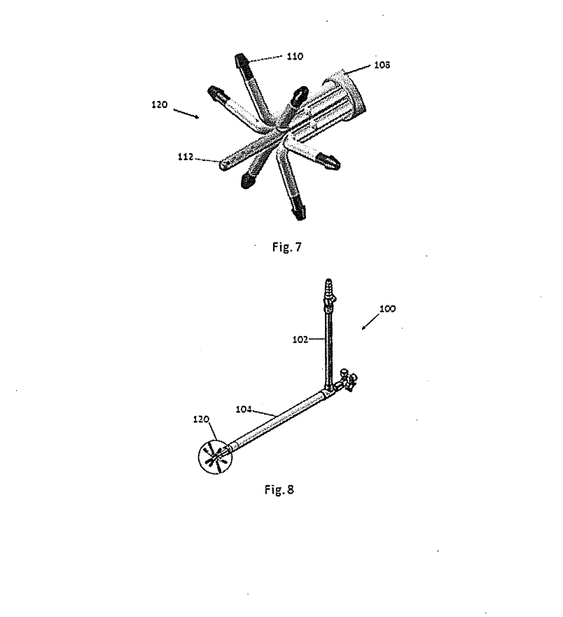

FIG. 7 provides a view of the manifold 108. The fluid supply lumen passes through a central opening in the manifold, and the distal tip of the centrally located fluid supply lumen can be seen 112. Also extending from the manifold are a plurality of six adaptors with tips 110.

The portion of the device shown in FIG. 7 is circled in FIG. 8 as the distal assembly 120. Also noted in the device 100 are the central and auxiliary tubes, 104 and 102, respectively.

Patients Amenable to Treatment:

Based on our studies and analysis, we have concluded that third-space fluids, including lymph, interstitial edema, and ascites released from capillary leakage of the gut, can be pro-inflammatory and play a critical role in the pathogenesis of secondary ARDS. Accordingly, the compositions and methods of the present invention can be used in any circumstance where pro-inflammatory fluids are accumulating in the abdominal cavity and/or peritoneal cavity. Both prophylactic and therapeutic treatments are within the scope of the invention. Prophylactic removal of inflammatory ascites is expected to prevent the development of ARDS or reduce the risk that a patient will develop ARDS (e.g., the progression of septic or hemorrhagic shock to ARDS). Employing MIST in already compromised patients (e.g., patients suffering from septic or hemorrhagic shock) is expected to be therapeutic. In particular instances, the patient may be one who has experienced trauma, developed sepsis or septic shock, has compromised renal function, or is experiencing hemorrhagic shock. Any of the methods of the invention can include a step of identifying a patient in need of treatment (e.g., identifying a patient who has experienced trauma sufficiently severe to place them at risk; a patient who has developed sepsis or septic shock; a patient who has compromised renal function; or a patient who is experiencing hemorrhagic shock). For example, one can use an imaging technique to detect accumulating and unwanted fluid in the abdomen. For example, abdominal ultrasonography is an effective means of localizing ascitic fluid (Hambridge et al., Radiographics 23:663-664 (2003)).

Sepsis is the leading cause of death in intensive care units and is defined on a continuum of disorders from sepsis (systemic inflammatory response (SIRS) with suspected infection) to severe sepsis (sepsis+organ dysfunction), septic shock (sepsis+refractory hypotension) and MODS (Russell, N. Engl. J. Med. 355:1699-1713 (2006); Angus and Wax, Crit. Care Med. 29:S109-116 (2001)). The present devices can be employ to treat a patient at any point in this continuum. Severe sepsis and septic shock carry mortality rates of 25-30% and 40-70%, respectively (Russell, N. Engl. J. Med. 355:1699-1713 (2006)). In gross terms, sepsis represents a maladaptive inflammatory procoagulant, and ultimately immunosuppressive interaction between host and infective pathogen(s). Animal models have elucidated complex signaling and metabolic pathways responsible for the characteristic changes in immune function, coagulation, fibrinolysis, cell death, tissue perfusion, and endocrine function during sepsis. Despite insight into these pathways, and improvements in monitoring, diagnostic modalities and resuscitative and ventilation strategies, novel therapies for sepsis have been remarkably slow to develop. To date, the only consensus approved therapeutic regimes are Early Goal Directed Therapy during the initial stages of disease, and Activated Protein C for severe sepsis (Russell, N. Engl. J. Med. 355:1699-1713 (2006); Dellinger et al., Crit. Care Med. 36:296-327 (2008); Rivers et al., N. Engl. J. Med. 345:1368-1377 (2001)).

The inherent difficulties in studying the critically ill population, enormous expense to carry out clinical trials and the complexity of the sepsis spectrum makes animal models essential tools to ultimately improve patient care. But animal models are also at fault for the lack of progress in developing effective sepsis treatments over the last twenty years. Indeed, multiple treatments have shown significant benefits in acute animal models only to worsen the outcome of patients in subsequent clinical trials (Calandra et al., J. Infect. Dis. 158:312-319 (1988); Fisher et al., N. Engl. J. Med. 334:1697-1702 (1996); McCloskey et al., Ann. Intern Med. 121:1-5 (1994)). As a result, many scientists have championed a critical re-evaluation of the criteria necessary for an animal model to truly replicate the complex pathogenesis of human sepsis (Dyson and Singer, Crit. Care Med. 37:S30-37 (2009); Piper et al., Crit. Care Med. 24:2059-2070 (1996); Opal and Patrozou, Crit. Care Med. 37:S10-15 (2009); Esmon, Crit. Care Med. 32:S219-222 (2004); Deitch, Shock 9:1-11 (1998)). These criteria include an animal species with similar anatomy and physiology to humans, a clinical setting utilizing standard clinical treatments such as fluid resuscitation, antibiotics and mechanical ventilation, the ability for repeated measurements, and an injury model that accurately reflects the etiology, pathogenesis and complexity of clinical severe sepsis and septic shock (Dyson and Singer, Crit. Care Med. 37:S30-37 (2009); Piper et al., Crit. Care Med. 24:2059-2070 (1996); Opal and Patrozou, Crit. Care Med. 37:S10-15 (2009); Esmon, Crit. Care Med. 32:S219-222 (2004); Deitch, Shock 9:1-11 (1998)).

Some, but not all, of the patients that develop septic or hemorrhagic shock progress to multiple organ dysfunction syndrome (MODS), a condition that is often lethal. Progression to MODS is dependent on several factors. The severity of the sepsis or hemorrhage, comorbidities, and patient genetics all play a role. An important factor in the development of MODS is exposure of the patient to a second insult or “hit.” For example, it has been shown that hemorrhagic shock in rats causes neutrophil priming but no lung or liver damage (Rezende-Nato et al., Shock 20:303-308 (2003)). If, however, the intra-abdominal pressure (IAP) is raised to levels simulating the abdominal compartment syndrome (ACS), these primed neutrophils become activated, causing both lung and liver damage (Rezende-Nato, supra). A corollary of this second-hit theory is that there is often an underlying driving force or “motor” that leads to the development of MODS.

Procedures for Deploying the Present Devices:

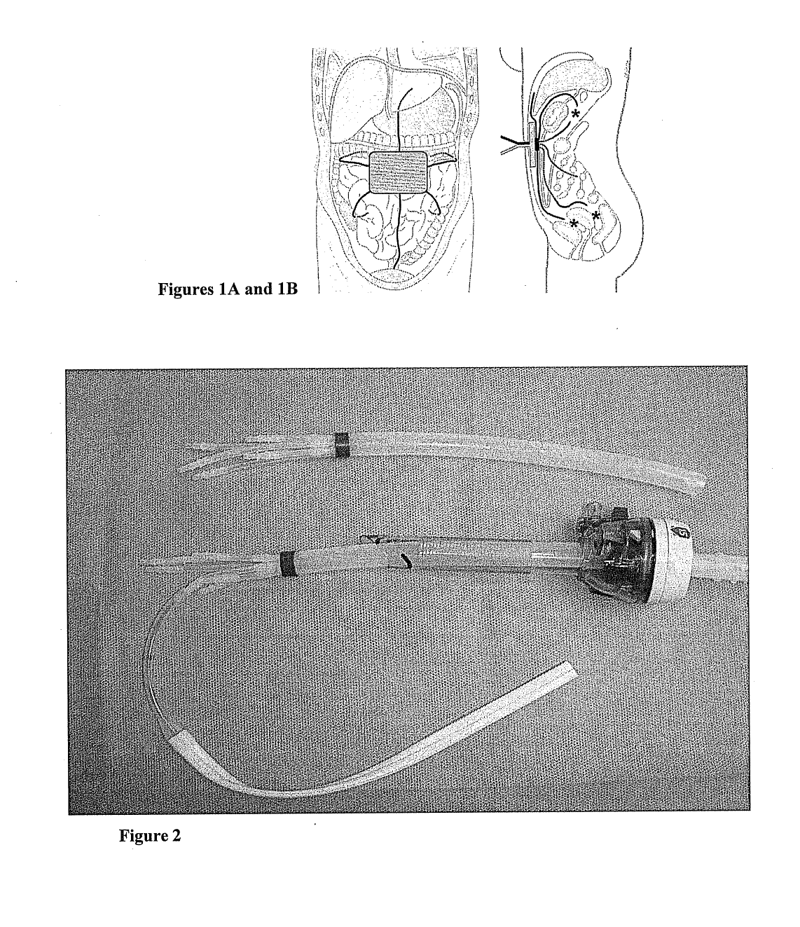

The devices of the invention can be surgically deployed in various ways. In one deployment, the most distal catheters can be deployed first and then attached, via the adaptors, to the fluid removal lumen within the central tube. For example, a plurality (e.g. 2-10) of catheters (e.g., BLAKE® drains) can be inserted into the peritoneal cavity through a trocar (a 15 mm trocar) followed by the distal end of a MIST device. In most instances, the device will be inserted until the manifold resides within the abdominal cavity. Using, for example, two laparoscopic clamps, the surgeon would then pick up the end of a catheter and a tip of one of the adaptors on the MIST device and push them together until the catheter is firmly attached. This process would be repeated until all of the catheters or drains are connected. Once connected, the surgeon would place the distal ends of all of the catheters in the dependent regions of the cavity to be treated (e.g., the regions of the peritoneal cavity as shown in FIGS. 1A and 1B. As noted, an advantage of the present devices is that they can be used to effect treatment through minimally invasive procedures. Thus, while the abdominal incision can be large, as occurs with a full laparotomy, the present methods can be carried out following only very small incision for laparoscopic insertion of the device. The laparoscopic device, such as a trocar, that is used to enter the abdomen can be only about 15 mm in diameter, so an incision of only about 15 mm would be required.

In another aspect, the invention features methods of reducing the risk of multiple organ dysfunction syndrome (MODS) or a condition that may precede MODS, such as septic shock. The methods can include the steps of: (a) providing a patient at risk (e.g., at risk for MODS); and (b) performing a minimally invasive surgical procedure that removes ascites fluid. The methods can also include a lavage; a sterile lavage fluid can be added to the peritoneal cavity in addition to simply removing the ascites fluid. The methods of the invention can be carried out with devices configured as described herein. For example, the surgical procedure can include the steps of: (a) inserting a plurality of catheters or drains through an abdominal incision such that the distal ends of the catheters are placed, at some time after the insertion, into anatomic recesses of the peritoneal cavity; (b) inserting a device as described herein into the abdominal cavity (optionally with the assistance of a guide, such as a trocar) and attaching the proximal ends of the catheters to the adaptors on the manifold; (c) attaching the proximal end of the central tube to a vacuum; and (d) applying, using the vacuum, negative pressure to the device. The anatomic recesses include the lesser sac, Morrison's pouch, pouch of Douglas, and pericolic gutters. The procedure that lavages the peritoneal cavity comprises delivering, under the force of gravity, a sterile fluid solution to the peritoneal cavity (e.g., a dialysis fluid, normal saline or a buffered saline solution). The patient's abdomen can be inflated with a physiologically acceptable gas to facilitate insertion of the plurality of catheters.

Administration of Therapeutic Agents:

The devices described herein are also uniquely suited for the topical application of therapeutic agents to the peritoneal cavity and organs. For example, the present devices can be used to deliver antimicrobial agents (e.g., an antibiotic, antiviral, or antifungal agent), a vasoactive agent, an anti-inflammatory agent, antiproteases, and other medications, or any combination thereof, to the peritoneal cavity and organs. Topical application of antibiotics and antiproteases has been shown in DPR to significantly aid in recovery by affecting endothelium permeability factors (Zakaria et al., Am. J. Surg. 5:443-448 (2003)). While a therapeutically effective amount of a therapeutic agent can be delivered in the context of fluid removal and/or lavage, the invention is not so limited. The devices described herein can be used to deliver therapeutic agents to patients whether or not they also have a need for fluid removal and/or lavage.

While the present devices were developed with the treatment of human patients in mind, the invention is not so limited. The devices and methods described herein can also be used in veterinary settings. For example, the devices and methods can be employed to treat household pets, such as dogs and cats, livestock, horses, non-human primates and other animals kept in captivity.

### EXAMPLES

### Example 1

### A Clinically Relevant Porcine Model of Sepsis

Although many sepsis treatments have shown efficacy in acute animal models, at present only activated protein C is effective in humans. The likely reason for this discrepancy is that most of the animal models used for preclinical testing do not accurately replicate the complex pathogenesis of human sepsis. Our objective in the study described below was to develop a clinically applicable model of severe sepsis and gut ischemia/reperfusion (I/R) that would cause multiple organ injury over a period of 48 hours.

The object of this study was to create a model of septic shock with true clinical relevance. Briefly, anesthetized, instrumented and ventilated pigs were subjected to a “two-hit” injury by placement of a fecal clot through a laparotomy and by claimping the superior mesenteric artery (SMA) for 30 minutes. Thus, our model combines intestinal ischemia and reperfusion with intraperitoneal infection. The animals were monitored for 48 hours. Wide spectrum antibiotics and intravenous fluids were given to maintain hemodynamic status. FiO₂ was increased in response to oxygen desaturation. Twelve hours following injury, a drain was placed in the laparotomy wound. Extensive hemodynamic, lung, kidney, liver, and renal function measurements and serial measurements of arterials and mixed venous blood gases were made. Bladder pressure was measured as a surrogate for intra-peritoneal pressure to identify the development of the abdominal compartment syndrome (ACS). Plasma and peritoneal ascites cytokine concentrations were measured at regular intervals. Tissues were harvested and fixed at necropsy for detailed morphometric analysis.

Polymicrobial sepsis developed in all animals. There was a progressive deterioration of organ function of the 48-hour period. The lung, kidney, liver and intestine all demonstrated clinical and histopathologic injury. Acute Lung Injury (ALI) and ACD developed by consensus definitions. Increases in multiple cytokines in serum and peritoneal fluid paralleled the dysfunction found in major organs.

The animal model of Sepsis+I/R replicates the systemic inflammation and dysfunction of the major organ systems that is typically seen in human sepsis and trauma patients. The model should be useful in deciphering the complex pathophysiology of septic shock as it transitions to end-organ injury and thus allow sophisticated preclinical studies on potential treatments. We believe the model will serve to generate detailed, reliable and unbiased pre-clinical data in support of treatments that, if successful in this model, would demonstrate a high likelihood of success in human clinical trials (Piper et al., Crit. Care Med. 24:2059-2070 (1996)).

Healthy female Yorkshire pigs (n=5, 22-30 kg) were pretreated with glycopyrrolate (0.01 mg/kg, intramuscular), Telazol™ (tiletamine hydrochloride and zolazepam hydrochloride (5 mg/kg, intramuscular)) and xylazine (2 mg/kg, intramuscular). A ketamine (3 mg/ml) plus xylazine (0.3 mg/ml) continuous infusion (3M model 3000 infusion pump) was used to maintain anesthesia at a rate of 100 mg/hr for the duration of the experiment. The rate was adjusted as needed to provide adequate anesthesia. All changes to the rate were recorded.

An open tracheostomy was performed and the animals connected to a Galileo™ ventilator (Hamilton Medical, Reno, Nev.). Initial settings were as follows: tidal volume (Vt) 12 cc/kg, respiratory rate (RR) of 15/min titrated to maintain PaCO₂ within the normal range. FiO₂ of 21% and positive end-expiratory pressure (PEEP) of 3 cm H₂O. Low tidal volume protective mechanical ventilation was not utilized since we did not want to protect the lung with the ventilator but rather measure the development of lung injury if it occurred.

Under sterile conditions, a left carotid artery catheter was placed for blood chemistry and gas content measurements (Roche Inc., Cobas b211) and systemic arterial pressure monitoring. A 4 cm right lateral neck incision was made, and a veinotomy performed on the right internal jugular vein for placement of a triple lumen catheter for anesthesia, fluid, and antibiotic administration. A right internal jugular Swan-Ganz catheter (7 French) was placed for measurement of pulmonary artery pressure (PAP) and pulmonary artery wedge pressure (PAW), sampling of mixed venous blood gases, and cardiac output (CO) (Agilent, CMS-2001). A Foley catheter was inserted into the bladder for measurement of urine output, collection of urine samples, and was connected to a pressure transducer leveled at mid-axillary line to measure bladder pressure (Pbladder).

A midline laparotomy was performed and the superior mesenter artery (SMA) was isolated and clamped for 30 minutes to induce intestinal ischemia. This was confirmed by the loss of the mesenteric pulse as well as the discoloration of the bowel. After 30 minutes, the clamp was removed and reperfusion was confirmed by the reappearance of the mesenteric pulse and return of normal color to the bowel. At this point, the cecum was brought out of the abdominal cavity and an enterotomy of 2 cm was performed to combine 0.55 cc/kg of feces with 2 cc/kg of blood obtained from the pig to create a fecal-blood clot. The cecum was returned to its anatomical position, with the enterotomy unclosed, and the clot was implanted into the right lower quadrant of the abdominal cavity. A catheter was placed in Morrison's pouch between the liver and right kidney and brought out through the body wall for collection of peritoneal fluids and sutured to the skin. The abdomen as then closed with 0-0 PDS sutures and the time recorded as T0 (i.e., zero hours following injury). The animals were then followed for twelve hours after injury (T12) at which time the mid-line incision was re-opened for abdominal decompression and allowed to drain passively. Collected ascites were flash frozen for measurement of inflammatory mediators. All of the animals were followed for a total of 48 hours or until the time of death.

All fluids were warmed to 37° C. in a water bath (Precision 280 Series, Thermo Electronic Corp). During the surgical preparation, pigs received a fluid bolus of lactated Ringers (1 liter, intravenously) over 30 minutes. Following TO measurements, broad-spectrum antibiotics (ampicillin 2 grams, intravenously (Bristol Myers Squibb, Princeton, N.J.) and Flagyl 500 mg, intravenously (Baxter, Deerfield, Ill.)) were delivered over 15 minutes. This antibiotic regimen was repeated at 12, 24, and 36 hours post injury. An intravenous infusion of lactated Ringers was administered at a rate necessary to maintain adequate volume status determined by urine output (UOP) and mean arterial pressure (MAP). Volume status was deemed inadequate if UOP decreased to less than 0.55 cc/kg/hr or if MAP decreased to less than 60 mmHg. All fluids infused or withdrawn were recorded to analyze fluid balance.

If arterial desaturation occurred (SaO₂ below 92%), FiO₂ was increased to maintain oxygenation. If 100% FiO₂ did not maintain adequate oxygenation, PEEP was increased (maximum PEEP allowed was 15 cm H₂O) in 2 cm H₂O increments until oxygenation was adequate or hemodynamics were comprised. If the animal triggered ventilations while fully anesthetized, Pancuronium bromide (0.1 mg/kg, intravenously) was given to control breathing.

ECG monitoring, pulse oximetry, mean arterial pressure (MAP), central venous pressure (CVP) pulmonary artery pressure (PAP), and pulmonary artery wedge pressure (PAW) were measured (Agilent, CMS-2001™ System M1176A with Monitor M1094B, Boebingen, Germany) using Edwards transducers (Pressure Monitoring Kit PXMK1183, Edwards Lifesciences). Cardiac output (CO) was measured by thermodilution (Agilent, CMS-2001™ System M1176A with Monitor M1094B, Boebingen, Germany). Three separate boluses of cold solution (dextrose 5% and sodium chloride 0.45%) were injected at end-expiration and the average of the three measurements was recorded. Physiologic measurements were made hourly (T0-T48).

Kidney function was assessed by measuring blood creatinine (Clinical Pathology Department at Upstate Medical University, using standard procedures) and BUN (Roche Cobras b221, Roche Diagnostics, Indianapolis, Ind.) levels at Baseline, every hour for the first six hours (BL, T0-T6) and every six hours thereafter. UOP was recorded hourly and samples of urine were flash frozen and T0, T12, T35 and T48 for measurement of protein concentration.

Alanine aminotransferase (ALT), aspartate aminotransferase (AST), total and direct bilirubin, albumin and total protein were measured by the Clinical Pathology Department at Upstate Medical University using standard procedures. Coagulation parameters (see below) were also measured as indicators of synthetic function of the liver.

Measurement of blood gases and chemistries were made with a Roche Blood gas analyzer (Cobras b221) at Baseline, every hour for the first six hours (BL-T6) and every six hours thereafter following injury. Both arterial and mixed venous samples were measured for pH, pCO₂, pO₂, SO₂, hematocrit, hemoglobin, sodium, potassium, chloride, ionized calcium, glucose, BUN, and lactate.

Prothrombin time (PT), international normalization ratio (IN), and activated partial thromboplastin time (PTT) was measured by the Clinical Pathology Department at Upstate Medical University using standard procedures.

A complete blood count (CBC) with differential including the white blood cell (WBC) and platelet count was done by the Clinical Pathology Department at Upstate Medical University using standard procedures.

To assess inflammatory mediators, blood was drawn, placed in sodium citrate tubes and spun at 3500 RPM at 15° C. for 10 minutes. The plasma was drawn off and snap-frozen in liquid nitrogen for inflammatory mediator analysis.

To collect peritoneal fluid (Pfluid), 20 ml of saline was injected into the catheter placed in the peritoneum and aspirated back into the syringe one minute later. The aspirate (saline plus ascites) was put into blud-topped tubes and spun at 3500 RPM at 15° C. for 10 minutes. The supernatant was drawn off and snap frozen for inflammatory mediator analysis.

To collect bronchoalveolar lavage fluid (BALF), at necroscopy, the right middle lobe was lavaged with 60 ml of normal saline (3 injections of 20 ml flushed into the right middle lobe bronchus and aspirated out) and the volume collected was recorded. The BALF was spun for 10 minutes at 3500 RPM at 15° C. The supernatant was drawn off and snap frozen for inflammatory mediator analysis.

Inflammatory mediators were measured in plasma and peritoneal fluid throughout the study and in the BALF at necropsy. Tumor necrosis factor alpha (TNF-α), IL-8, IL-6, IL-1β, IL-12, transforming growth factor beta (TGF-β) (R&D Systems, Minneapolis, Minn.), IL-10 (Immuno-biological laboratories, Minneapolis, Minn.), were measured using pig specific ELISA assays according to the manufacturer's assigned specifications. Endotoxin levels were assessed using an end point chromogenic LAL assay (Lonza Group, Ltd., Basel, Switzerland). Lastly, blood was collected in standard vials for aerobic and anaerobic bacteria identification. Mediators sampled in plasma are presented as pg protein per milliliter of plasma. Mediators in Pfluid and BALF were normalized to the total protein present in the sample and presented as pg/ng.

The heart and lungs were removed en bloc. Gross photographs were made after the lungs were inflated to peak airway pressure of 25 cm H₂O. The heart was then removed and the bronchus to the right middle lobe was exposed. The right mainstem bronchus was clamped, the left lung was filled with 10% neutral buffered formalin, and the trachea was clamped. The lung was then immersed in formalin for a minimum of 48 hours before processing for histology. Specimens of dependent lung areas were obtained by measuring 3 cm medial from the aortic groove and making a longitudinal section. Two samples were removed from the most medial section 3 cm from the distal tip of the lung.

For kidney histopathology, the organ was divided along the central axis and specimens of the cortex and adjacent medulla were obtained and fixed in 10% buffered formalin for a minimum of 48 hours.

For intestinal histopathology, sections of proximal (10 cm from the gastic pylorus), mid-(estimated mid-jejunum), and distal (10 cm from ileocecal junction) small intestine were excised and fixed in 10% buffered formalin for a minimum of 48 hours.

For liver histopathology, a 3 cm section of liver was harvested from the center of the left lobe and fixed in 10% buffered formalin. All sections were stained with hematoxylin and eosin. Each slide was appraised microscopically and representative histological characteristics were photographed at low, medium, and high magnification (4×, 10× and 40× objectives).

Organ edema was measured using the W/D ratio=(wet weight/dry weight) for all four organs. Tissue from the lung, intestine, liver, and kidney was excised, minced, immediately weighed and placed in an oven at 60° C. and allowed to dry. Dry weight was determined when the weight did not change over a 24 hour period. The wet/dry weights from unpublished historic naïve control animals (n=4) were used as comparison.

All data were expressed as mean±SE. A repeated measures ANOVA was used to determine differences over time. A student's t-test was used to assess differences in the wet/dry weight ratio of experimental subjects compared to historical controls. Significance was assumed if the probability of the null hypothesis was less than 5% (p<0.05). All analyses were performed on version 5.0.1.2 of JMP (SAS Institute, Cary, N.C.).

We found no measurable co-morbidities in any animal at the beginning of the experiment. Bacteremia and polymicrobial sepsis was noted in all animals with positive blood cultures for Pseudomonas aeruginosa, Escherichia coli, Klebsiella pneumoniae, and Proteus mirabilis. Mortality before T48 was 60%.

Table 1 summarizes the changes in hemodynamic and lung function. Hypotension and hypodynamic shock was evidenced by significant decreases in mean arterial pressure and cardiac output. Acute lung injury (ALI) developed as defined by consensus conference definition with a P/F ratio below 300 without evidence of left ventricular failure (Bernard et al., J. Crit. Care 9:72-81 (1994)). Lung injury was also demonstrated by significant increases in Ppeak, Pplat and decreased Cstat, and confirmed by histological assessment showing congested capillaries and interstitial granulocytic infiltration. Focal alveolar atelectasis with dilated alveolar ducts and alveolar spaces contained fibrinous deposits suggestive of proteinaceous infiltrate. Pbladder rose to greater than 20 mmHg with evidence of lung failure, indicating the development of ACS according to consensus conference definition (Malbrain et al., Acta Clin. Belg. Suppl. 7:44-59 (2007)). The lung wet-to-dry ratio was significantly higher than our historic naïve controls. No macroscopic indications of barotraumas were observed.

TABLE 1
Hemodynamic and Pulmonary Function

| | BL (n = 5*) | T6 (n = 5*) | T12 (n = 5*) | T24 (n = 3*) | T36 (n = 2*) | T48 (n = 2*) | p < 0.05 |
|---|---|---|---|---|---|---|---|
| HR | 131.4 ± 7.8 | 83.4 ± 5.5 | 75.8 ± 7.9 | 71.3 ± 7.2 | 77.5 ± 4.5 | 76.0 ± 8.0 | § |
| MAP | 122.4 ± 4.9 | 69.4 ± 3.2 | 66.4 ± 3.7 | 62.0 ± 1.2 | 61.5 ± 1.5 | 51.0 ± 80 | § |
| PAP | 26.2 ± 1.59 | 27.0 ± 2.2 | 29.8 ± 2.6 | 24.7 ± 2.7 | 26.0 ± 3.0 | 31.5 ± 4.5 | |
| CVP | 8.8 ± 1.2 | 6.6 ± 1.2 | 9.6 ± 1.0 | 8.0 ± 1.7 | 7.0 ± 1.0 | 12.0 ± 1.0 | |
| CO | 4.61 ± 0.63 | 2.74 ± 0.67 | 1.76 ± 0.22 | 1.72 ± 0.18 | 1.86 ± 0.23 | 1.81 ± 0.11 | § |
| Pbladder | 11.2 ± 1.2 | 12.0 ± 1.9 | 21.4 ± 2.8 | 17.0 ± 2.5 | 18.0 ± 3.0 | 21.0 ± 3.0 | |
| Temp | 36.02 ± 0.42 | 35.1 ± 0.44 | 35.8 ± 0.33 | 35.13 ± 0.12 | 35.8 ± 0.0 | 35.8 ± 0.05 | |
| Ppeak | 18.2 ± 1.0 | 19.6 ± 1.0 | 21.0 ± 1.0 | 25.3 ± 3.5 | 22.5 ± 0.5 | 30.5 ± 3.5 | § |
| Pplat | 16.6 ± 0.8 | 17.8 ± 1.0 | 19.8 ± 0.8 | 22.7 ± 2.9 | 21.0 ± 0.0 | 28.5 ± 3.5 | § |
| Pmean | 6.7 ± 0.5 | 6.9 ± 0.4 | 7.1 ± 0.2 | 8.2 ± 1.4 | 7.6 ± 0.6 | 12.0 ± 1.0 | |
| Cstat | 24.6 ± 0.6 | 23.2 ± 0.9 | 20.8 ± 1.8 | 19.9 ± 2.3 | 19.5 ± 0.9 | 13.9 ± 0.5 | § |
| PaCO₂ | 29.3 ± 1.9 | 30.2 ± 2.9 | 28.6 ± 3.1 | 30.6 ± 4.0 | 35.3 ± 6.4 | 30.2 ± 2.5 | |
| PaO₂ | 110.23 ± 12.03 | 109.4 ± 6.32 | 130.23 ± 26.37 | 205.27 ± 28.3 | 147.27 ± 55.57 | 149.15 ± 19.65 | § |
| P/F | 524.9 ± 57.3 | 521.0 ± 30.1 | 503.2 ± 29.8 | 488.23 ± 43.3 | 281.6 ± 123.8 | 233.6 ± 104.1 | § |

MAP = mean arterial pressure (mmHg),
PAP = pulmonary artery pressure (mmHg),
CVP = central venous pressure (mmHg),
CO = cardiac output (L/min),
Pbladder = bladder pressure (mmHg),
Temp = core temperature (C.°),
Ppeak = peak airway pressure (cmH₂O),
Pplat = plateau pressure (cmH₂O),
Pmean = mean airway pressure (cmH₂O),
PaCO₂ = arterial carbon dioxide partial pressure (mmHg),
PaO₂ = arterial oxygen partial pressure (mmHg),
P/F = PaO₂/fraction of inspired oxygen (FiO₂).
Data are expressed as mean ± SEM.
§ = p < 0.05 following RM ANOVA

Table 2 shows changes in blood composition over the course of the study. There were severe declines in hemoglobin, platelets, total protein and albumin. Percent hematocrit showed statistically significant changes and WBC fluctuated considerably over the course of the study but did not change significantly with respect to time.

TABLE 2
Blood Composition

| | BL (n = 5*) | T6 (n = 5*) | T12 (n = 5*) | T24 (n = 3*) | T36 (n = 2*) | T48 (n = 2*) | p < 0.05 |
|---|---|---|---|---|---|---|---|
| Hgb | 11.22 ± 0.34 | 12.86 ± 0.4 | 11.44 ± 0.48 | 10.68 ± 0.41 | 8.83 ± 2.32 | 9.70 ± 0.10 | § |
| Hct | 34.50 ± 0.93 | 39.68 ± 1.26 | 34.70 ± 1.42 | 32.54 ± 1.44 | 27.17 ± 7.26 | 30.15 ± 0.15 | § |
| WBC | 12.66 ± 1.79 | 7.62 ± 2.58 | 8.2 ± 3.14 | 7.76 ± 2.54 | 8.10 ± 3.96 | 6.55 ± 1.65 | |
| Platelets | 400.0 ± 56.1 | 349.5 ± 47.1 | 168.7 ± 16.6 | 188.6 ± 46.7 | 155.7 ± 70.2 | 133.0 ± 48.0 | § |
| Total Protein | 5.4 ± 0.19 | 3.54 ± 0.23 | 2.82 ± 0.06 | 2.38 ± 0.16 | 2.37 ± 0.20 | 2.15 ± 0.15 | § |
| Albumin | 3.66 ± 0.14 | 2.44 ± 0.07 | 1.94 ± 0.06 | 1.68 ± 0.12 | 1.37 ± 0.23 | 1.25 ± 0.15 | § |

Hgb = hemoglobin,
Hct = hematocrit,
WBC = white blood cell count (KμL⁻¹),
Platelets (KμL⁻¹),
Total Protein (g dL⁻¹),
Albumin (g dL⁻¹).
Data are expressed as mean ± SEM.
§ = p < 0.05 following RM ANOVA

Tables 3 and 4 summarize the changes in kidney and liver function and coagulation. BUN significantly increased over the course of the study and creatinine doubled over the course of the experiment, indicative of acute renal injury. The average hourly urine output dropped significantly over the course of the study despite fluid resuscitation. This could be due to a continual rise in intra-abdominal pressure assessed by bladder pressure, which reached values over 20 mmHg. Histopathologic exam of the kidneys showed areas of early cortical tubular atrophy accompanied by interstitial and perifascular edema. There was also loss of tubular architecture with epithelial sloughing, but no injury was seen in the glomeruli. Similar but less severe injury was noted in the renal medulla. The W/D ratio of the kidney was not significantly different than historic controls. Aspartate aminotransferase (AST) increased but without reaching significance. No significant changes were seen in bilirubin or alkaline phosphatase levels. International Normalized Ratio (INR), prothrombin time (PT) and partial thromboplastin time (PTT) rose to noteworthy levels from the clinical perspective but were not statistically significant. Histopathologic assessment of the liver unveiled conspicuous interstitial edema in the connective tissues of the portal areas and lobular septa, and heavy leukocyte infiltration was frequently observed in these edematous areas. Hepatic lobules were commonly marked by peripheral congestion of the sinusoids and distinct paracentral necrosis. Loss of cellular architecture was local and limited to the proximity of the central vein. The W/D ratio was not significantly different than the historic control.

TABLE 3
Kidney Function

| | BL (n = 5*) | T6 (n = 5*) | T12 (n = 5*) | T24 (n = 3*) | T36 (n = 2*) | T48 (n = 2*) | p < 0.05 |
|---|---|---|---|---|---|---|---|
| UOP | 126.0 ± 23 | 30.6 ± 7.7 | 54.0 ± 25.3 | 70.3 ± 30.1 | 60.0 ± 30.0 | 31.5 ± 11.5 | |
| BUN | 8.52 ± 0.65 | 10.81 ± 0.82 | 12.58 ± 1.62 | 16.77 ± 2.82 | 24.37 ± 5.43 | 33.0 ± 5.0 | § |
| Creatinine | 1.00 ± 0.0 | 1.00 ± 0.10 | 1.07 ± 0.12 | 1.35 ± 0.45 | 1.65 ± 0.65 | 2.10 ± 0.80 | |

UOP = urine output (ml/hr),
BUN = blood urea nitrogen (mg dL⁻¹),
Creatinine (mg dL⁻¹).
Data are expressed as mean ± SEM. § = p < 0.05 following RM ANOVA

TABLE 4
Liver Function Panel and Coagulation

| | BL (n = 5*) | T6 (n = 5*) | T12 (n = 5*) | T24 (n = 3*) | T36 (n = 2*) | T48 (n = 2*) | p < 0.05 |
|---|---|---|---|---|---|---|---|
| AST | 34.0 ± 5.9 | 32.0 ± 6.3 | 41.6 ± 7.9 | 109.6 ± 54.0 | 41.7 ± 0.7 | 114.5 ± 77.5 | |
| ALT | 35.2 ± 3.4 | 26.2 ± 2.3 | 23.8 ± 2.8 | 25.6 ± 3.1 | 19.7 ± 6.6 | 27.0 ± 5.0 | |
| ALK PHOS | 208.8 ± 25.8 | 164.4 ± 21.8 | 168.0 ± 19.8 | 179.8 ± 28.1 | 175.7 ± 64.0 | 205.0 ± 26.0 | |
| PT | 13.22 ± 0.10 | 14.66 ± 0.53 | 15.76 ± 0.49 | 18.12 ± 1.10 | 28.50 ± 11.42 | 19.35 ± 2.15 | |
| PTT | 43.88 ± 5.22 | 39.30 ± 3.90 | 40.80 ± 4.77 | 32.32 ± 3.88 | 32.03 ± 6.09 | 22.80 ± 1.30 | |
| INR | 0.998 ± 0.009 | 1.14 ± 0.5 | 1.25 ± 0.48 | 1.49 ± 0.11 | 2.66 ± 1.28 | 1.63 ± 0.23 | |

AST = aspartate aminotransferase (U L⁻¹),
ALT = alanine amiontransferase (U L⁻¹),
ALK PHOS = alkaline phosphatase (U L⁻¹),
PT = prothrobin time (sec),
PTT = partial thromboplastin time (sec),
INR = international normalization unit.
Data are expressed as mean ± SEM.

Without reliable clinical indications of intestinal injury, histopathology is the strongest evidence available to assess disease state. Pathology was most prominent in the mucosa, with loss of the surface epithelium, flattening of denuded villi, and sloughing of the lamina propria onto the intestinal lumen. There was also a high incidence of congestion of small blood capillaries in the upper compartment of the mucosa, which was suggestive of hypoxia associated with poor blood flow through the end-capillary bed. The submucosa was grossly edematous and marked by prominently dilated lymph vessels. The cellularity and edema present in the serosa were typical of acute peritonitis. The W/D ratio was significantly greater than that of historic control animals.

Table 5 summarizes the blood chemistry data. Arterial pH showed significant changes over time, as did arterial lactate, which rose sharply during the acute phase of injury, returned to normal with resuscitation, then rose again at the end of the study. Systemic oxygenation, as assessed by venous oxygen saturation (SvO₂) significantly decreased. Potassium significantly increased over the course of the study. Decreases were seen in sodium and chloride but they were not significant. Blood glucose levels also decreased throughout the experiments.

TABLE 5
Blood Chemistry

| | BL (n = 5*) | T6 (n = 5*) | T12 (n = 5*) | T24 (n = 3*) | T36 (n = 2*) | T48 (n = 2*) | p < 0.05 |
|---|---|---|---|---|---|---|---|
| pH | 7.483 ± 0.020 | 7.412 ± 0.019 | 7.473 ± 0.026 | 7.500 ± 0.020 | 7.366 ± 0.088 | 7.404 ± 0.05 | § |
| Lactate | 2.75 ± 0.13 | 6.23 ± 0.49 | 1.93 ± 0.38 | 1.77 ± 0.23 | 4.67 ± 2.47 | 3.95 ± 1.75 | § |
| SvO₂ | 81.6 ± 10.4 | 63.4 ± 3.5 | 46.3 ± 4.3 | 44.8 ± 3.5 | 53.0 ± 6.7 | 44.2 ± 8.4 | § |
| Glu | 100.5 ± 15.4 | 90.0 ± 10.8 | 69.3 ± 2.9 | 48.3 ± 3.8 | 44.0 ± 12.1 | 49.5 ± 13.5 | § |
| Na⁺ | 149.9 ± 5.0 | 141.7 ± 1.9 | 143.7 ± 2.0 | 139.2 ± 1.4 | 142.3 ± 1.6 | 137.1 ± 0.1 | |
| K⁺ | 3.22 ± 0.07 | 3.63 ± 0.15 | 4.23 ± 0.22 | 4.28 ± 0.22 | 4.93 ± 0.64 | 4.82 ± 0.22 | § |
| Cl⁻ | 115.95 ± 3.67 | 112.40 ± 1.22 | 114.83 ± 2.03 | 108.67 ± 2.68 | 113.07 + 1.67 | 113.80 ± 2.30 | |
| Ca²⁺ | 1.05 ± 0.04 | 1.18 ± 0.04 | 1.06 ± 0.07 | 4.60 ± 3.60 | 1.02 ± 0.01 | 0.99 ± 0.04 | |

pH = arterial pH,
Lactate = plasma lactate concentration (mmol L⁻¹),
SvO₂ = venous oxygen saturation (%),
Glu = serum glucose concentration (mg dL⁻¹),
Na⁺ (mmol L⁻¹),
K⁺ (mmol L⁻¹),
Cl^{- (mmol L}⁻¹),
Ca²⁺ (mmol L⁻¹).
Data are expressed as mean ± SEM.
§ = p < 0.05 following RM ANOVA

Table 6 summarizes the inflammatory mediator data in the ascites and plasma. A significant change in several cytokines was observed in the peritoneal ascites fluid (Pfluid). TNF-α, IL-1β, IL-6, IL-8 and IL-12 had significantly higher levels at the end of the study as compared with baseline values. Systemically, TNF-α and IL-1β were significantly elevated and IL-12 significantly decreased. TNF-α, IL-1β, IL-6, IL-8, IL-10, and IL-12 were all present in the BALF.

TABLE 6
Cytokines

| | | | | | | | | p > |
|---|---|---|---|---|---|---|---|---|
| | Source | BL (n = 5*) | T6 (n = 5*) | T12 (n = 5*) | T24 (n = 3*) | T36 (n = 2*) | T48 (n = 2*) | 0.05 |
| TNF-α | Plasma | 92.1 ± 14.1 | 235.1 ± 48.1 | 191.7 ± 34.9 | 239.0 ± 87.0 | 631.2 ± 337.3 | 852.8 ± 79.1 | § |
| | (pg/mL) | | | | | | | |
| | PFluid | 0.0092 ± 0.0073 | N/A | 0.364 ± 0.135 | 0.741 ± 0.341 | 0.861 ± 0.051 | 0.837 ± 0.010 | § |
| | (pg/ng) | | | | | | | |
| | BALF | | | | | | 0.104 ± 0.103 | |
| | (pg/ng) | | | | | | | |
| IL-8 | PFluid | 0.00278 ± 0.002 | N/A | 1.513 ± 0.374 | 3.593 ± 0.937 | 4.761 ± 0.892 | 4.749 ± 1.381 | § |
| | (pg/ng) | | | | | | | |
| | BALF | | | | | | 0.255 ± 0.068 | |
| | (pg/ng) | | | | | | | |
| IL-6 | Plasma | 39.1 ± 0.0 | 4814.6 ± 1715.4 | 4404.4 ± 22445.0 | 1401.1 ± 789.6 | 23702.0 ± 21565.8 | 8728.0 ± 3394.7 | |
| | (pg/mL) | | | | | | | |
| | PFluid | 0.0125 ± 0.003 | N/A | 1.790 ± 0.421 | 1.669 ± 0.441 | 1.073 ± 0.226 | 1.488 ± 0.302 | § |
| | (pg/ng) | | | | | | | |
| | BALF | | | | | | 0.539 ± 0.094 | |
| | (pg/ng) | | | | | | | |
| IL-1β | Plasma | 230.8 ± 0.0 | 120.8 ± 49.6 | 146.2 ± 32.2 | 157.4 ± 39.2 | 389.3 ± 151.5 | 424.6 ± 60.3 | § |
| | (pg/mL) | | | | | | | |
| | PFluid | 0.0355 ± 0.0332 | N/A | 0.433 ± 0.078 | 1.260 ± 0.301 | 2.580 ± 0.341 | 1.121 ± 0.142 | § |
| | (pg/ng) | | | | | | | |
| | BALF | | | | | | 0.493 ± 0.130 | |
| | (pg/ng) | | | | | | | |
| IL-12 | Plasma | 540.0 ± 93.4 | 319.4 ± 42.4 | 190.3 ± 43.0 | 176.0 ± 21.5 | 380.6 ± 74.2 | 403.1 ± 93.9 | § |
| | (pg/mL) | | | | | | | |
| | PFluid | 0.00954 ± 0.0038 | N/A | 0.0656 ± 0.0249 | 0.144 ± 0.022 | 0.133 ± 0.065 | 0.133 ± 0.021 | § |
| | (pg/ng) | | | | | | | |
| | BALF | | | | | | 0.000947 ± 0.000021 | |
| | (pg/ng) | | | | | | | |
| IL-10 | Plasma | 31.5 ± 0.0 | 163.1 ± 102.2 | 32.0 ± 4.0 | 113.7 ± 56.8 | 40.4 ± 2.9 | 77.2 ± 0.0 | |
| | (pg/mL) | | | | | | | |
| | PFluid | 0.00032 ± 0.000086 | N/A | 0.0155 ± 0.0082 | 0.0387 ± 0.0051 | 0.0124 ± 0.0123 | 0.0239 ± 0.008 | |
| | (pg/ng) | | | | | | | |
| | BALF | | | | | | 0.00094 ± 0.00002 | |
| | (pg/ng) | | | | | | | |
| TGF-β | Plasma | 2815.1 ± 346.4 | 3138.9 ± 416.3 | 2315.5 ± 573.2 | 2822.1 + 813.6 | 2199.5 ± 941.5 | 1935.7 ± 758.7 | |
| | (pg/mL) | | | | | | | |
| | PFluid | 0.0323 ± 0.0122 | N/A | 0.0737 ± 0.0265 | 0.110 ± 0.0495 | 0.132 ± 0.0345 | 0.167 ± 0.0889 | |
| | (pg/ng) | | | | | | | |
| | BALF | | | | | | 0.5575 ± 0.2843 | |
| | (pg/ng) | | | | | | | |

TNF-α = tumor necrosis factor,
IL = interleukin-8,-6,-1β,-12,-10,
TGF-β = transforming growth factor beta.
Data are expressed as mean ± SEM.
§ = p < 0.05 following RM ANOVA

Although animal models have been indispensable in advancing our understanding of the sepsis spectrum, the distinction must be made between experimental models that are not clinically applicable and those that bear clinical relevance and are therefore “translatable” to human trials. Multiple preclinical studies of potential sepsis treatments have shown promising results in acute animal models, but were ineffective or even increased mortality when tested in human clinical trials. The likely explanation for this discrepancy is that the animal models used did not replicate the complex pathogenesis of human sepsis and thus the drugs tested were not treating the same disease that occurs in humans (Calandra et al., J. Infect. Dis. 158:312-319 (1988); Fisher et al., N. Engl. J. Med. 334:1697-1702 (1996); McCloskey et al., Ann. Intern Med. 121:1-5 (1994)). Multiple authors have commented on the necessary components of an ideal model of sepsis (Dyson and Singer, Crit. Care Med. 37:S30-37 (2009); Piper et al., Crit. Care Med. 24:2059-2070 (1996); Opal and Patrozou, Crit. Care Med. 37:S10-15 (2009); Esmon, Crit. Care Med. 32:S219-222 (2004); Deitch, Shock 9:1-11 (1998); Deitch, Shock 24 Suppl 1:19-23 (2005); Scachtrupp and Wilmer, Acta Clinica Belgica 1:225-232 (2007); Buras et al., Nat. Rev. Drug Discov. 4:854-865 (2005)). Our model fulfills most of these criteria, utilizing an animal with similar anatomy and physiology to humans, inclusion of standard clinical procedures (e.g., fluid resuscitation, mechanical ventilation, antibiotic therapy), a protracted time course that mimics clinical reality, and relevant etiology. Most importantly, our results are consistent with the systemic inflammation and multiple organ injury that are the hallmarks of sepsis pathophysiology.

Acute lung injury (ALI) was evidenced functionally by a significant decrease in static compliance and P/F ratio with an increase in peak and plateau pressures. Histopathology was consistent with acute lung injury and there was a significant increase in lung water as compared with Controls. The alveolar collapse, lymphocytic accumulation, and hyaline membrane formation are similar to autopsy findings in human patients that died from severe sepsis and septic shock (Lucas, Current Diagnostic Pathology 13:375-388 (2007)). The presence of TNF-α, IL-1β, IL-6, IL-8, and IL-10 in BALF is also consistent with findings in human ARDS patients (Pugin et al., Am. J. Respir. Crit. Care Med. 153:1850-1856 (1996); Park et al., Am. J. Respir. Crit. Care Med. 164:1896-1903 (2001); Armstrong and Millar, Thorax 52:442-446 (1997)). Combined, these data demonstrate that this model causes an insidious onset of acute lung injury typical of ARDS and is consistent with the known human literature (Shimada et al., Chest 76:180-186 (1979); Ware and Matthay, N. Engl. J. Med. 342:1334-1349 (2000)).

There was a rise in both BUN and creatinine, consistent with human septic shock; a rising creatinine level is a known late manifestation of acute renal injury in human sepsis (Khadaroo and Marshall, Crit. Care Clin. 18:127-141 (2002)). The delayed renal (as compared to lung) dysfunction was expected and typical of the sequence of organ injury that occurs in human sepsis. Fewer inflammatory cells reside in the kidney and thus parenchymal cell damage secondary to inflammation is minimal or protracted (Wang and Ma, Am. J. Emerg. Med. 26:711-715 (2008)). Urine output in our animals dropped precipitously early in the study and remained considerably lower than baseline even with fluid resuscitation. A similar time course is seen in septic humans where early oliguria is the results of reduced renal perfusion and continues as a result of evolving renal injury (Khadaroo and Marshall, Crit. Care Clin. 18:127-141 (2002)). Renal histopathology was limited mainly to the convoluted tubules of the cortex, along with some interstitial and perivascular edema. However, the glomeruli were not affected. Minimal renal histopathology is typical in patients with septic shock and demonstrated that renal histopathologic injury does not reflect the severity of the kidney injury (Hotchkiss et al., Crit. Care Med. 27:1230-1251 (1999)). The concept of “cell stunning” has been used to describe this phenomenon and perhaps represents a shift towards a more perfunctory state of cell function in response to sepsis (Hotchkiss and Karl, N. Engl. J. Med. 348:138-150 (2003)). Thus, our clinical, inflammatory, and histopathologic findings demonstrate the similarity in both injury and time course between our model and acute renal failure seen in human septic shock (Hotchkiss et al., Crit. Care Med. 27:1230-1251 (1999); Hotchkiss and Karl, N. Engl. J. Med. 348:138-150 (2003); Wan et al., Crit. Care Med. 36:S198-203 (2008)).

Our injury model produced only moderate changes in liver synthetic function and moderate clinical injury as measured by AST/ALT. There was a significant decrease in serum albumin and total protein and an elevated AST and INR. These findings are similar to those in the first phase of liver dysfunction seen in human septic shock (Dhainaut et al., Crit. Care Med. 29:S42-47 (2001)). This first phase of primary hepatic dysfunction in sepsis is a result of hepatosplanchnic hypoperfusion and can be blunted or reversed with adequate fluid support (Dhainaut et al., Crit. Care Med. 29:S42-47 (2001)). The elevated serum lactate level at the final reading may be due to this primary hepatic dysfunction, resulting in decreased lactate clearance. The increase in INR suggests a diminished hepatic production of coagulation factors. Reduced plasma albumin concentration may be due to decreased liver synthesis or loss due to leakage of plasma proteins into the interstitium secondary to increased capillary permeability (Khadaroo and Marshall, Crit. Care, Clin. 18:127-141 (2002); Moshage et al., J. Clin. Invest. 79:1635-1641 (1987)). Minimal histopathology was seen in the liver, similar to septic patients where hepatic cell injury and liver dysfunction are common, however histopathologic liver damage has been shown to be limited and nonspecific (Lucas, Current Diagnostic Pathology 13:375-388 (2007)). Thus, our findings are consistent with pathology seen in septic patients, typically showing little inflammation with prominent Kuppfer and endothelial cells, and centriacinar necrosis (Lucas, Current Diagnostic Pathology 13:375-388 (2007)). In summary, the liver dysfunction present in our animal model closely represents the findings seen in human patients undergoing currently accepted fluid resuscitation regimens.

Overall, the cytokine response seen in out study was consistent with that seen in human septic shock. The patterned rises in TNF-α, and IL-6, and the decline in IL-12, mirrored the patterns documents in patients with severe sepsis and septic shock (Cohen, Nature 420:885-891 (2002); Damas et al., Ann. Surg. 215:356-362 (1992); Bozza et al., Crit. Care 11:R49 (2007); Emmanuilidis et al., Shock 18:301-305 (2002)). In addition, IL-10 was detectable in peritoneal fluid and BALF, consistent with results from clinical trials demonstrating the immunosuppressive phase of septic shock associated with organ injury (Bozza et al., Crit. Care 11:R49 (2007); Dhainaut et al., Crit. Care Med. 33:341-348 (2005)).

Cytokines are also implicated in the coagulation abnormalities and vascular endothelial activation that play a major role in the development or organ dysfunction (Dhainaut et al., Crit. Care Med. 33:341-348 (2005). Cytokine-induced consumption of anticoagulant proteins and suppression of the fibrinolytic system during sepsis results in deposition of fibrin clots and microcirculatory dysfunction (Cohen, Nature 420:885-891 (2002); Amaral et al., Intensive Care Med. 30:1032-1040 (2004); Ince, Crit. Care 9 Suppl 4:S13-19 (2005)). We observed evidence of consumptive coagulopathy from laboratory results showing prolonged PT, PTT, and elevated INR, as well as histopathological evidence of microthrombosis in all organs. Furthermore, activated platelets participate in the formation of microvascular clots, provide a surface for further coagulation activation, and release inflammatory mediators propagating the inflammatory response (Levi, Hematology 10 Suppl 1:129-131 (2005)). The resultant consumption of platelets likely contributes to the marked thrombocytopenia commonly seen in critically ill and septic patients (Levi, Hematology 10 Suppl 1:129-131 (2005)). All the animals in our model had a significant precipitous decrease in their platelet count throughout the study.

Currently, there is no way to measure acute intestinal dysfunction secondary to septic shock at the bedside. However, at necropsy we found significant intestinal edema, a large volume of ascites and extensive histopathologic injury, characterized by denudated villi and epithelial sloughing. Recent work by Malbrain and De Laet has stressed the concept of the gut as a “motor” of organ injury secondary to septic shock and cautioned about being deterred from this idea because of the difficulty assessing gut function (Malbrain and De Laet, Crit. Care Med. 37:365-366 (2009)). We demonstrated a significant increase in inflammatory peritoneal fluid and intestinal edema, enough to cause ACS, which likely contributed to injury in other organs. Thus, our model provides evidence of acute intestinal injury that likely contributes to worsening prognosis for severe sepsis and septic shock patients. Shah et al. recently demonstrated that a combination of hemorrhagic shock, mesenteric venous hypertension and crystalloid resuscitation lead to the development of ACS in a clinically relevant porcine model (Shah et al., J. Trauma 68:682-689 (2010)). This was the first reported physiologic animal ACS model; our model is the second to create ACS in an etiologically relevant manner, via sepsis and gut ischemia/reperfusion.

In summary, we have developed a clinically accurate, chronic animal model of septic shock that replicates the dysfunction seen in the major organ systems of humans being treated for sepsis. The time course of organ dysfunction was also typical of sepsis pathogenesis in humans. The combination of infected clot plus ischemia/reperfusion injury simulates the two injuries most likely to cause shock in humans (i.e., sepsis and trauma). The model fits most of the criteria necessary for a clinically applicable animal that will yield “good evidence” that a treatment effective in this model will also be effective in humans.

### Example 2

### Peritoneal Negative Pressure Therapy for the Prevention of Multiple Organ Injury in a Porcine Model of Sepsis

The studies below were carried out to test our hypothesis that peritoneal negative pressure therapy (NPT) could reduce systemic inflammation and organ damage. Briefly, pigs (n=12) were anesthetized and surgically instrumented for hemodynamic monitoring. Through a laparotomy, the superior mesenteric artery was clamped for 30 minutes. Feces was mixed with blood to form a fecal clot that was placed into the peritoneum, and the abdomen was closed. All subjects were treated with standard isotonic fluid resuscitation, wide spectrum antibiotics, and mechanical ventilation (essentially as described in Example 1) and were monitored for 48 hours. Animals were separated into two groups 12 hours (T12) after injury. For NPT (n=6), an abdominal wound vacuum dressing was placed in the laparotomy and negative pressure (−125 mmHg) was applied (T12-T48). As a control (n=6) we allowed passive drainage (PD). NPT removed a significantly greater volume of ascites (860±134 mL) than did passive drainage (88±56 mL). Systemic inflammation (e.g., TNF-α, IL-1β, IL-6) was significantly reduced in the NPT group and was associated with significant improvement in intestine, lung, kidney, and liver histopathology. Our data suggest NPT efficacy is partially due to an attenuation of peritoneal inflammation by the removal of ascites. However, the exact mechanism needs further elucidation. The clinical implication of this study is that sepsis/trauma can result in an inflammatory ascites that may perpetuate organ injury; removal of the ascites can break the cycle and reduce organ damage.

As noted above, the current study was carried out using the porcine model described in Example 1. With regard to treatment for sepsis, a computerized number generator was used to randomly divide the animals into two groups; an NPT-treated group and a passive drainage group. For the first 12 hours of the protocol, the animals were treated in an identical fashion. All animals received a regimen of intravenous fluids and antibiotics at a dose and quantity established in our initial experiments. Pulmonary and arterial blood pressure and gases, cardiac output, UOP, heart rate, IAP, and temperature were continually monitored and recorded every 60 minutes. Ischemia-reperfusion and the placement of a fecal-blood clot were carried out as described above. A catheter was placed in Morrison's pouch between the liver and right kidney and brought out through the skin for collection of peritoneal ascites. This catheter was sutured to the skin in a purse-string fashion. Collected ascites was flash-frozen for measurement of inflammatory mediators. The abdomen was then closed with sutures and the time recorded as T0.

The entire abdomen was reopened at T12, and the V.A.C. Abdominal Dressing System (KCl, Inc., San Antonion, Tex.) was applied to the open wound as per packet instructions. The fenestrated bioinert plastic that housed the sponge material was in direct contact with the intestine. However, to prevent iatrogenic bowel injury, care was taken to ensure that the dressing sponge did not touch the bowel. For animals in the control group, the dressing was placed but the vacuum was not activated (i.e., no negative pressure was applied). However, the drain line was left open to allow passive drainage of ascites. In the experimental group, the dressing was placed and the vacuum was activated so that negative pressure (−125 mmHg) was applied continuously for the remainder of the experiment.

Hemodynamic measurements and calculations were made as described above; fluids and antibiotics were administered as described above, as was mechanical ventilation. Inflammatory mediators were measured in the plasma, peritoneal ascites fluid (Pfluid) and bronchoalveolar lavage fluid (BALF). The heart and lungs were removed at the conclusion of the experiment en bloc, and prepared for histological analysis as described above. Tissue edema was assessed as described above, using the ratio of tissue wet weight to dry weight.

We found that lung compliance was significantly improved with NPT as compared with passive drainage. Animals in the NPT group had significantly lower Pmean, peak inspiratory pressures, and plateau pressures than animals in the passive drainage group. PaO₂, PaCO₂, SaO₂, and SvO₂ were not different between the groups. Oxygenation expressed as a P—F ratio was higher in the NPT group, albeit not significantly different from that in the passive drainage group (P=0.0812). The mortality rate in the NPT group was 17% versus 50% in the passive drainage group, but this difference was not statistically significant (P=0.1859). Negative pressure therapy demonstrated a significant improvement in cardiac output and PAW. Minimal differences were seen between groups in HR, MAP, or PAP. Urine output was significantly lower in the passive drainage group as compared with the NPT group despite the fact that more fluids were given to the former group. A significantly elevated IAP (measured via the bladder pressure) in the passive drainage group as compared with the NPT group may have been one of the mechanisms contributing to the reduced UOP. There was a significant difference in AST and albumin by treatment×time in the NPT group compared with the PD, with little change in alkaline phosphatase. Alanine aminotransferase decreased to a similar degree in each group.

We describe intestinal injury solely on necropsy results. There was a significant reduction in intestinal edema (W-D) in the NPT as compared with the passive drainage group (PD=7.44±0.46; NPT=5.97±0.45, P<0.05). There were no significant differences in W-D in the other organs (lung, liver, kidney). There was also a significant improvement in histopathology in the NPT as compared with the PD group. Both dependent and nondependent sections of the lung showed a significant decrease in atelectasis, fibrinous deposits, and leukocyte infiltration when NPT was used. Negative pressure therapy also caused a significant decrease in all kidney and intestine mucosal parameters assessed, with the exception of Gruenhagen spaces, as well as a significant decrease in edema in the intestinal wall. In the liver, NPT led to significant decreases in interstitial edema, hepatocellular necrosis, and interstitial WBCs. Histology of the intestine was internally consistent with no sign of localized injury. This was in agreement with the gross observation that there were no signs of damage or fistula formation to the bowel that was in contact with the fenestrated bioinert plastic VAC dressing.

In peritoneal ascites fluid, negative pressure therapy significantly reduced IL-6 and IL-8. There were no significant differences between groups in vWf, CRP, TGF-β, PGE2, endotoxin, antioxidants, C5A, or IL-10. In plasma, there was significant reduction in TNF-α, IL-12, IL-6, and IL-1β with NPT as compared with the PD group. No significant differences were found between the group in vWf, CRP, TGF-β, PGE2, endotoxin, and antioxidants.

An important finding of this study was that NPT reduced histologic damage to the lungs, intestine, kidney, and liver. The results suggest that the mechanism for this protection involved removal of inflammatory peritoneal ascites causing a moderation of systemic inflammation (SIRS), which diminished end organ damage. Although NPT significantly reduced the histopathology in all organs measured, a concomitant improvement in organ function cannot be conclusively asserted. The mean values for lung function did meet our criteria for ALI in the PD group; however, there was variability within both groups, with 66% of the animals in the PD group meeting ALI criteria and 33% of the animals in the NPT group. This variability combined with the small number of animals does not allow us to draw the conclusion that NPT reduced ALI. These data also suggest that histologic injury is a very sensitive predictor that is manifest before the organ becomes clinically dysfunctional. We speculate that some organ failure would have occurred if the experiment had been performed for another 24 hours.

### Example 3

### In Vitro Simulations

To test the ability of model devices to collect fluid, we made fluid solutions of varying viscosity and tested their uptake from an open basin and in a setting that more closely resembles fluid in the abdomen. To vary the viscosity of the solutions, we used orange juice with different amounts of pulp, and in some tests we also included oatmeal. To simulate fluid buildup in the intestines, we filled plastic tubes with water, which served as a model of the intestines, and placed these tubes in a sealed canister along with tubes of the manifold, clamped at their ends, and water. The sealed canister was placed under vacuum pressure at −125 mmHg to simulate a partial vacuum environment. In all cases, upon attaching suction to the model device, the test fluids were extracted, usually at an essentially linear rate, from the basin or simulated intestine.

A number of embodiments of the invention have been described. Nevertheless, it will be understood that various modifications may be made without departing from the spirit and scope of the invention. Accordingly, other embodiments are within the scope of the following claims.

What is claimed is:

1. A medical device for removing fluid from a bodily cavity, the device comprising a central tube having a proximal end configured to receive an external vacuum source and a distal end comprising a manifold from which a plurality of adaptors extend, the adaptors having tips to which one or more catheters suitable for placement in the bodily cavity can be attached.

2. The device of claim 1, wherein the central tube comprises a rigid or semi-rigid material and the manifold has a diameter that is at least or about as large as the combined diameters of the plurality of adaptors.

3.-4. (canceled)

5. The device of claim 1, wherein the central tube is cylindrical or polygonal, the manifold is cylindrical or polygonal, and the manifold has, around the periphery of the cylinder or the polygon, three to eight openings from which three to eight adaptors extend.

6. (canceled)

7. The device of claim 1, wherein the manifold is the integral distal portion of the central tube, the central tube having a bottom surface and a plurality of openings in the peripheral wall or bottom surface from which adaptors extend.

8. The device of claim 7, wherein the adaptors extending from the bottom surface of the manifold vary in length relative to one another and/or are angled to project away from the central line of the central tube.

9. The device of claim 1, further comprising a plurality of catheters attached to the tips of the plurality of adaptors.

10. The device of claim 9, further comprising an external sleeve concentric to the central tube into which the plurality of catheters can be retracted.

11. The device of claim 1, wherein the central tube comprises a first lumen having a proximal end configured to receive an external fluid source and a distal end having at least one opening for introducing the external fluid into the bodily cavity, wherein the first lumen is parallel to, adjacent to, or concentric inside or outside of a second lumen and is positioned relative to the central tube such that the distal end of the first lumen extends beyond the manifold.

12. The device of claim 11, wherein the first lumen has a valve proximal to its proximal end.

13. The device of claim 9, wherein at least one of the plurality of catheters is perforated along its length.

14. The device of claim 9, wherein at least one of the plurality of catheters has an inflatable balloon around the periphery of its distal end.

15. The device of claim 9, wherein the plurality of catheters comprise polyurethane and have one or more of the following characteristics: an outer diameter of about 5-10 mm; a thickness of about 1-2 mm; an inner diameter of about 3-9 mm; a vacuum rating of about 600-800 mmHg; and a length of about 5-25 cm.

16. The device of claim 1, wherein the central tube comprises a first lumen and a second lumen, the first lumen being aligned with the central tube along the entire length of the first lumen and the second lumen being aligned with the central tube along a distal portion of the second lumen and misaligned with the central tube along a proximal portion of the second lumen.

17. The device of claim 11, wherein the second lumen comprises, at its proximal end, a sampling port.

18.-30. (canceled)

31. A kit comprising the medical device of claim 9 and instructions for use.

32. A kit comprising the medical device of claim 1 and one or more of: a plurality of catheters adapted for attaching to the tips of the plurality of adaptors of the medical device; a sterile fluid; a syringe adapted for attaching to a sampling port of the medical device; and a therapeutic agent.

33. The kit of claim 32, wherein the sterile fluid is suitable for lavaging the abdominal cavity or for delivering the therapeutic agent.

34. The kit of claim 32, wherein at least one of the plurality of catheters is perforated along its length.

35. The kit of claim 32, wherein at least one of the plurality of catheters has an inflatable balloon around the periphery of its distal end.

36. The device of claim 16, wherein the second lumen comprises, at its proximal end, a sampling port.
